SEQUENCE LISTING (3 sequences)

SEQ ID NO: 1 - protein, 12 aa, Artificial Sequence
  Synthetic
  MOD_RES (1)..(1): H-Se-CH2-CH2-CO-
  MOD_RES (4)..(5): OBzl
  MOD_RES (12)..(12): -NH2
TYICEVEDQKEE

SEQ ID NO: 2 - protein, 12 aa, Artificial Sequence
  Synthetic
SSLTLAPFSWSL

SEQ ID NO: 3 - protein, 12 aa, Artificial Sequence
  Synthetic
TYICEVEDQKEE

(19) United States
(12) Patent Application Publication
(10) Pub. No.: US 2024/0238428 A1
(43) Pub. Date: Jul. 18, 2024

(54) TARGETED SELENIUM CONJUGATES AS COUNTERMEASURES FOR VIRAL AND CELLULAR PATHOGENS

(71) Applicant: Texas Tech University System, Lubbock, TX (US)

(72) Inventors: Ted W. Reid, Wolfforth, TX (US); Joe Fralick, Lubbock, TX (US); Phat L. Tran, Lubbock, TX (US); Nicholas Bergfeld, Lubbock, TX (US); Olof Sundin, Lubbock, TX (US)

(21) Appl. No.: 18/559,821

(22) PCT Filed: May 11, 2022
(86) PCT No.: PCT/US2022/028695

§ 371 Date: Nov. 9, 2023

Related U.S. Application Data
(60) Provisional application No. 63/186,901, filed on May 11, 2021.

(51) Int. Cl. A61K 47/64; A61K 33/04; A61K 47/54; A61P 31/04; A61P 31/14; A61P 31/18

(52) U.S. Cl. CPC A61K 47/64; A61K 33/04; A61K 47/554; A61P 31/04; A61P 31/14; A61P 31/18

(57) ABSTRACT

The present invention includes composition and methods for making and using a selenium-carrier conjugate for in vivo administration comprising: covalently attaching a selenium compound that reacts in vivo with naturally occurring reduced thiols and oxygen to produce superoxide, which can include a Fenton Complex that allows for a generation of hydroxyl radicals, and that wherein the selenium-carrier conjugate is stabilized against enzymatic degradation and clearance from the body and the synthetic targeting carrier binds target.

### CROSS-REFERENCE TO RELATED APPLICATIONS

This application claims priority to U.S. Provisional Application Ser. No. 63/186,901, filed May 11, 2021, the entire contents of which are incorporated herein by reference.

### TECHNICAL FIELD OF THE INVENTION

The present invention relates in general to the field of targeted selenium conjugates as countermeasures for viral and cellular pathogens.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on May 10, 2022, is named TECH2157WO_ST25.txt and is 2 kilobytes in size.

### BACKGROUND OF THE INVENTION

Without limiting the scope of the invention, its background is described in connection with treatment with selenium.

One example is U.S. Pat. No. 9,987,304, issued to Sutich, entitled “Method and topical composition for the treatment of Rosacea and skin erythema using selenium sulfide”, in which the inventor is said to teach a method for treating facial Rosacea and skin erythema using selenium sulfide as the sole active ingredient in a topically applied administration to a user's face in combination with an inactive moisturizing ingredient.

Another example is U.S. Patent Publication No. 20190388463, filed by Kenyon, et al., entitled “Pharmaceutical Composition for Treating Cancer Comprising Trypsinogen and/or Chymotrypsinogen and an Active Agent Selected from a Selenium Compound, a Vanilloid Compound and a Cytoplasmic Glycolysis Reduction Agent”. These applicants are said to teach pharmaceutical compositions and methods of treating cancer with pharmaceutical composition for treating cancer comprising trypsinogen and/or chymotrypsinogen and an active agent selected from a selenium compound, a vanilloid compound and a cytoplasmic glycolysis reduction agent.

What is needed are new compositions and method for the targeted delivery of agents that can be used to treat infections (e.g., bacterial, fungal, protozoan) and diseases or conditions such as cancer, in which a selenium compound is used to target superoxide or perhydroxyl radicals at a target site, while reducing unwanted side effects and non-specific damage to cells or tissues.

### SUMMARY OF THE INVENTION

In one embodiment, the present invention includes a method of making a selenium-carrier conjugate for in vivo administration comprising: covalently attaching a selenium compound that reacts in vivo with naturally occurring reduced thiols and oxygen to produce superoxide and perhydroxyl radicals to a synthetic targeting carrier to form the selenium-carrier conjugate, wherein the selenium-carrier conjugate is stabilized against enzymatic degradation and clearance from the body and the synthetic targeting carrier binds target. In one aspect, the synthetic targeting carrier binds with high specificity to an external domain of a targeted protein or membrane protein on the surface of an animal virus, cellular microbe, or mammalian cell. In another aspect, the selenium-carrier conjugate produces superoxide radicals and their protonated derivative, the perhydroxyl radical, that locally damages lipids in viral envelopes or proteins in viral capsids to inactivate animal viruses or kill one or more bacteria, fungi, protozoans, cancerous or otherwise disease-causing mammalian cells and selectively kills infected cells infected with the one or more membrane-enveloped viruses. In another aspect, the synthetic targeting carrier for selenium is a chemically modified peptide or peptidomimetic molecule that specifically binds to the targeted protein, membrane protein, carbohydrate or other biological macromolecular assembly. In another aspect, wherein a peptide precursor of the synthetic peptidomimetic targeting carrier is designed by expressing and displaying a phage expression library of peptides having different amino acid sequence permutations, then selecting phage expressing peptides with the desired properties, expressing permuted amino acid peptide sequence within the polypeptide chain or the surface protein of a bacteriophage protein, and selecting the bacteriophage that bind to the targeted protein on the surface of one or more viruses or cells, or selecting completely random permutations of an amino acid peptide sequence or a partially random amino acid peptide sequence permutation encoded in the genome of and displayed as part of a protein of the corresponding capsid surface of a bacteriophage, or selecting a binding assay using a bacteriophage library. In another aspect, the method further comprises selecting peptide precursor sequences that bind at positions within a few nanometers of a membrane of the one or more membrane-enveloped viruses or cells infected with the one or more membrane-enveloped viruses to enhance the efficiency of virus inactivation or infected cell killing by their selenium-carrier conjugates. In another aspect, the method further comprises modifying the peptide precursor sequence originally selected by bacteriophage display or another molecular display method, wherein the step of modifying comprises at least one of: extending the n-terminus and c-terminus by adding chemical groups that hinder the action of terminal peptidases; coupling carbohydrate polymers that increase solubility and extend the lifetime of the selenium-carrier complex: changing the amino acid sequence, chemically modifying the amino acids, substituting one or more peptide linkages, or substituting the peptide chain with one or more D-amino acids, wherein the modifications enhance binding affinity of the modified peptide precursor to the target and increase its resistance to proteolysis in vivo (e.g., the peptide has SEQ ID NO: 1 or 2), to increase clinical effectiveness of the selenium-carrier conjugate. In another aspect, the selenium-carrier conjugate is a catalytically active selenium-carrier conjugate, wherein binding and dissociation rate constants of the catalytically active selenium-carrier conjugate attaches transiently to the target site, wherein the seleno-conjugate detaches and reattaches multiple times, allowing the catalytically active selenium-carrier conjugate to destroy multiple viral or cellular targets, either in vitro or in vivo. In another aspect, the target is at least one of: one or more membrane-enveloped viruses is a coronavirus, influenza virus, human immunodeficiency virus (HIV), respiratory syncytial virus (RSV), herpesvirus or other membrane-enveloped human or animal virus. Also included as targeted virus particles are protein-encapsidated non-envelope viruses. These include viruses of the following genera and species: Adenovirus. Poliovirus. Enterovirus. Rhinovirus, Hepatitis A virus. Yellow fever virus. West Nile virus. Dengue virus. Zika virus. Hepatitis C virus. Rotavirus. Papillomavirus. In another aspect, the method further comprises selecting selenium-carrier conjugates that selectively kill virally-infected cells by binding to excess virally-encoded envelope proteins on the plasma membrane surface during viral replication. In another aspect, the selenium is specifically targeted to exposed surfaces of cells for the purpose of killing these cells, wherein the cells are selected from bacteria, fungi, or protozoans, abnormal human, infected human, or mammalian cells such as cancers, or dysfunctional cells. In another aspect, the selenium-carrier conjugate is stored as a relatively stable and inactive diselenide dimer of the form R—Se—Se—R or R—Se—Se—R′, at a suitable ambient temperature that could range from −20° ° C., to 40° C. In another aspect, the method further comprises administering the inactive diselenide dimer intravenously, orally, topically, nasally, or through pulmonary administration by inhalation of an aqueous mist or dry powder to epithelia of the upper and lower respiratory tract, wherein the inactive diselenide dimer is converted to superoxide-generating R—Se—H monomers by in situ glutathione and other reducing compounds. In another aspect, the peptide has SEQ ID NO: 1 or 2.

In another embodiment, the present invention includes a method of treating a human or animal patient after viral exposure or during an active viral infection comprising: covalently attaching a synthetic targeting carrier specific for a target with a selenium compound to form a selenium-carrier conjugate; providing the selenium-carrier conjugate by intravenous, nasal, oral, topical or pulmonary administration to the human or animal patient; and wherein the selenium-carrier conjugate reacts in vivo with naturally occurring thiols and oxygen at the target to catalytically generate short-lived superoxide and perhydroxyl radicals. In one aspect, the selenium-carrier conjugate binds with high specificity to an external domain of a targeted membrane protein on a surface of a membrane-enveloped animal virus or a surface plasma membrane of a virus-infected cell. In one aspect, the selenium-carrier conjugate generates superoxide radicals that transform into perhydroxyl radicals at an acidic membrane interface or a polyanionic environment with protein-encapsidated viruses. In another aspect, the perhydroxyl radicals cause oxidative damage to membrane lipids of a virus to render its protective membrane permeable, or disrupted, and to further inactivate the virus by damage to at least one of: viral proteins, viral RNA or DNA, or a viral genome. In another aspect, the method further comprises providing the human or animal patient with a sufficient amount of the selenium-carrier conjugate, as formulated for in vivo administration, to effectively reduce the levels of active virus and viral infection. In another aspect, the synthetic targeting carrier is a peptidomimetic molecule that specifically binds to a targeted viral envelope membrane protein. In another aspect, a peptide precursor of the peptidomimetic synthetic targeting carrier is made by: expressing and displaying a peptide having an amino acid sequence permutation on a surface of a bacteriophage library, and selecting the bacteriophage that bind to a targeted transmembrane protein on a surface of one or more membrane-enveloped viruses. In another aspect, the method further comprises the step of modifying a peptide to form a modified peptide precursor by at least one of: changing the amino acid sequence, chemical modifying the amino acids, substituting one or more peptide linkages, or substituting with one or more D-amino acids, wherein the modifications at least one of: enhance binding affinity of the modified peptide precursor to the target, increase resistance to proteolysis, or increase an effectiveness of the selenium-carrier conjugate in vivo (e.g., the peptide has SEQ ID NO: 1 or 2). In another aspect, the target is one or more membrane-enveloped viruses selected from a coronavirus, influenza virus, human immuno-deficiency virus (HIV), respiratory syncytial virus (RSV), or other membrane-enveloped virus. In another aspect, the target is at least one of: encapsidated non-envelope animal viruses selected from genera and species: Adenovirus, Poliovirus, Enterovirus, Rhinovirus, Hepatitis A virus, Yellow fever virus, West Nile virus, Dengue virus, Zika virus, Hepatitis C virus, Rotavirus, Papillomavirus, wherein if a virus is a non-enveloped viruses, production of the effective perhydroxyl radical is entirely dependent on the presence of protein and nucleic acid polyanions.

In another embodiment, the present invention includes a method of treating a human or animal patient infected with a cellular microbial pathogens selected from a bacteria, fungi or protozoa comprising: forming a selenium-carrier conjugate by covalently attaching to a selenium compound with a synthetic targeting carrier specific for the bacteria, fungi or protozoa; providing the selenium-carrier conjugate by intravenous, nasal, oral, topical or pulmonary administration of said carrier with a sufficient amount of the selenium-carrier conjugate, as formulated for in vivo administration, to effectively reduce the levels of actively proliferating pathogen; reacting the selenium-carrier conjugate in vivo with naturally occurring thiols and oxygen to catalytically generate short-lived superoxide and perhydroxyl radicals; wherein the selenium-carrier conjugate binds with high specificity to an external domain of a targeted cell membrane protein on the surface of an actively growing and proliferating cell, or a relatively inactive cyst-like form of a cellular microbe; wherein the attached selenium-carrier conjugate generates superoxide radicals that transform into perhydroxyl radicals at the acidic plasma membrane interface, and wherein the perhydroxyl radicals cause at least one of: sufficient oxidative damage to plasma membrane lipids to render the cell permeable and cause lysis, or sufficient damage to bacteria, fungi or protozoa DNA through mutations or chromosomal breaks to prevent significant replication of the bacteria, fungi or protozoa. In one aspect, the synthetic targeting carrier is a peptido-mimetic molecule that specifically binds to at least one of: a targeted pathogen-encoded cellular plasma membrane protein or a specific protein located on a surface of a spore or cyst form of the pathogen, or a cell wall constructed of carbohydrate and carbohydrate-peptide polymers that are specific binding targets for the selenium-carrier conjugate. In another aspect, a peptide precursor of the peptidomimetic synthetic targeting carrier is made by: expressing and displaying a library of peptides having different amino acid sequence permutations on a surface of a bacteriophage, and selecting the bacteriophages that bind to the targeted transmembrane protein on a surface of the one or more membrane-enveloped viruses. In another aspect, the method further comprises the step of modifying a selected peptide precursor by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo (e.g., the peptide has SEQ ID NO: 1 or 2). In another aspect, the selenium-carrier conjugate kills or permanently inactivates actively growing, inactive, latent or spore-like forms of bacteria. In another aspect, the selenium-carrier conjugate kills or permanently inactivates fungal infections, wherein the fungi and opportunistic pathogens of the genera Histoplasma, Pneumocystis, Coccidiomyces, Candida. In another aspect, the selenium-carrier conjugate kills or permanently inactivates protozoal infections with selenium-carrier conjugates would work by killing protozoal pathogens selected from Plasmodium falciparum, Trypanosoma cruzi, or Entamoeba histolytica.

In another embodiment, the present invention includes a method of treating a human or animal patient with a cancer or other disorder caused by abnormal cells comprising: covalently attaching a synthetic targeting carrier to a selenium compound to form a selenium-carrier conjugate, wherein the synthetic targeting carrier specifically binds to a target on the cancer cell or abnormal cell; and administering the selenium-carrier conjugate by intravenous, nasal, oral or pulmonary administration to the human or animal patient, wherein the selenium-carrier conjugate reacts in vivo with naturally occurring thiols and oxygen to catalytically generate many short-lived superoxide radicals to treat the cancer or other disorder caused by abnormal cells. In another aspect, the abnormal cells are abnormal immune cells that cause an autoimmune disorder selected from multiple sclerosis, lupus or Graves' disease. In another aspect, the synthetic targeting carrier is a peptido-mimetic molecule that specifically binds to the targeted cellular plasma membrane protein specific to a cancer cell or another abnormal, disease-causing cell of the patient. In another aspect, a peptide precursor of a peptidomimetic synthetic targeting carrier is made by: expressing and displaying a library of peptides having different amino acid sequence permutations on the surface of a bacteriophage, and selecting the bacteriophages that bind to the targeted transmembrane protein on the surface of the one or more membrane-enveloped viruses, wherein the synthesized peptide is attached to Se. In another aspect, the method further comprises the step of modifying a selected peptide precursor by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo. In another aspect, the method further comprises the step of selectively ablating specific defective cell types to create niches for replacement cells prior to a stem cell-based cell replacement therapy.

In another embodiment, the present invention includes an antiviral, antibacterial, antifungal, antiprotozoal, anticancer or anti-abnormal human or animal cell method, the method comprising: targeting a first and a second target of the virus, bacterial, protozoa, cancer or abnormal cell with a first and a second selenium-carrier complex, wherein: the first target is a viral, bacterial, protozoan, cancer, or abnormal cell targeted by the first selenium-carrier complex; and the second target is targeted by the second selenium-carrier complex, wherein the Fenton complex is selected from: an organometallic compound or conjugate containing one or more atoms of iron, copper or other transition metals that catalyzes a conversion of superoxide, perhydroxyl radical or hydrogen peroxide into other compounds, that generate hydroxyl radicals and reactive oxygen species; wherein at least a portion of the Fenton Complex is located close to the selenium-carrier complex that catalyzes conversion of selenium-generated superoxide, perhydroxyl radical or hydrogen peroxide into hydroxyl radicals. In one aspect, the highly reactive hydroxyl radicals are generated locally from superoxide, perhydroxyl radical, or hydrogen peroxide by metal atoms of the Fenton Complex to chemically modify at least one of: membrane lipids, polypeptides, RNA, DNA, carbohydrates or other biological molecules. In another aspect, the first and a second selenium-carrier are administered in vivo simultaneously or sequentially. In another aspect, the Fenton complex is iron, copper or other transition metal covalently attached to, tightly chelated by, coordinated with or enclosed by an organic molecule. In another aspect, the organic molecule is a synthetic metal-coordinating compound, or a siderophores coupled to the peptide or a peptidomimetic targeting molecule. In another aspect, the organo-metallic compound is chemically modified or extended to produce an organic molecule that binds to RNA. DNA or both by intercalate between the bases. In another aspect, the intercalating organo-metallic compound is membrane-permeable to enveloped viruses to targeting production of hydroxyl radicals and RNA or DNA damaging inactivating and mutagenic effects on the membrane-enveloped virus particles. In another aspect, the intercalating organo-metallic compound is membrane-impermeable, such that the Fenton complex will preferentially bind to a genetic material of a protein-encapsidated non-enveloped virus particle to target production of hydroxyl radicals to a genetic material of virus. In another aspect, the method further comprises chemically linking to a peptide or peptidomimetic compound to bind with high selectivity to a protein that is a binding target of the peptide to target selenium to the surface of the virus. In another aspect, the viral protein or other molecule that selectively binds to the selenium-carrier complex exists as a dimer, trimer, or higher level multimer, wherein superoxide-generating selenium atoms are positioned within nanometers of Fenton complex metal atoms that convert to highly reactive hydroxyl radicals. In another aspect, the second selenium-carrier complex of the Fenton complex is directed to a viral binding site different from the peptide or peptidomimetic synthetic carrier that is conjugated to the first selenium-carrier complex. In another aspect, the first or the second selenium-carrier complex selectively binds to a different, non-competing site on the same target viral protein molecule. In another aspect, the Fenton complex and the first selenium-carrier complex selectively bind to two different proteins localized to a plasma membrane domain of a virus-infected cell, viral envelope or viral capsid. In another aspect, the first and second selenium-carrier complexes are formulated for in vivo administration to permit a higher dosage of a relatively lower toxicity iron-based Fenton complex in conjunction with a smaller dosage of the potentially more toxic selenium conjugate, when compared to unconjugated selenium. In another aspect, the first target is a first viral surface protein, and the second target is a viral membrane envelope or capsid protein. In another aspect, the Fenton complex is an iron-filled ferritin complex containing roughly 4500 iron atoms brought into the proximity of the active selenium-conjugate by a targeting peptide, peptidomimetic, protein or antibody. In another aspect, the Fenton complex is constructed by using a chemical linker to covalently cross-link a single ferritin complex to a targeting peptide, peptidomimetic, protein or antibody. In another aspect, a virus and cell-targeting carrier for the Fenton complex is a peptide, protein, antibody or peptidomimetic further comprising an antibody or peptide that binds selectively to human ferritin protein (e.g., the peptide has SEQ ID NO: 1 or 2). In another aspect, the ferritin is from autologous human plasma. In another aspect, the first and second targets are pathogens selected bacteria, fungi and protozoans. In another aspect, the Fenton complex is an organometallic compound or conjugate containing one or more atoms of iron, copper or other transition metals that catalyze conversion of superoxide, perhydroxyl radical or hydrogen peroxide into hydroxyl radicals and other reactive oxygen species. In another aspect, the Fenton complex metal atoms are highly reactive and chemically modify molecules, including membrane lipids, polypeptides, RNA, DNA, carbohydrates and other biological molecules. In another aspect, the cancer cells and other abnormal, dysfunctional cells, wherein the first and second selenium-carrier complexes have an increased effect, range, specificity of action, and are synergistic when used in combination comparted to each used individually. In another aspect, the cancer cells and other abnormal, dysfunctional cells comprise a single surface protein or combinations of cell surface proteins that are distinctive to the cancer cell. In another aspect, the cancer cells and other abnormal, dysfunctional cells are killed when they are pre-cancerous, senescent, inappropriately secrete signaling molecules, or are otherwise dysfunctional. In another aspect, the abnormal cells are immune cells that cause an autoimmune disease, an autoinflammatory disease or an allergy. In another aspect, the peptide has SEQ ID NO: 1 or 2. In another aspect, the organometallic compound is:

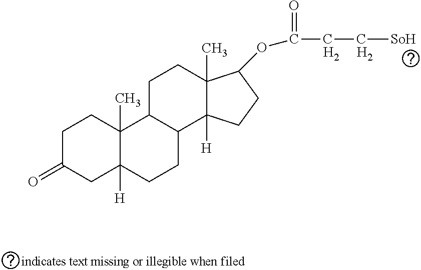

### BRIEF DESCRIPTION OF THE DRAWINGS

For a more complete understanding of the features and advantages of the present invention, reference is now made to the detailed description of the invention along with the accompanying figures and in which:

FIG. 1 shows the sequence of a peptide mixed with the viral isolate and allowed to stand for one hour in the presence of glutathione (250 uM) of SEQ ID NO:1 (Thr Tyr Ile Cys Glu Val Glu Asp Gln Lys Glu Glu).

FIG. 2 is a graph that shows effectiveness at a very low concentration of seleno-peptide (0.1 ug/ml) resulted in 95% inactivation of HIV-1 (this was measured by the ability of the infected cells to produce virus).

FIG. 3 is a graph that shows effectiveness against prostate cancer cell lines and found that it will kill both PC-3 and DU-145 cells.

FIG. 4 is a graph that shows the effect of the selenium-steroid conjugate and selenium plus steroid (not conjugated) on normal prostate cells, colorectal cancer cells, and normal human lung cells.

FIG. 5 is a graph that shows the inactivation of HIV virus infectivity with an anti-CD4 12-mer selenopeptide.

FIG. 6 is a graph that shows the effect of selenium-attached phage #8 on the viability of E. coli XL1-blue/pYPR1 under added oxygen.

FIG. 7 is a graph that shows the effect of seleno-peptide #8 (10 microM) on the E, coli XL1-blue/pYPR1 strain, with or without glutathione, under added oxygen.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention provides a method for optimizing the design and use of a selenium-carrier conjugate of the form R—Se—Se—R and R—Se—H, wherein R is an organic compound consisting of a small organic molecule linked to a protein, synthetic peptide, chemically modified peptide or peptidomimetic compound. This conjugate is designed to bind selectively to a protein or carbohydrate integral to the surface of a virus or cellular pathogen such as a bacterium, fungus or protozoan. It may also be used to remove a range of dysfunctional human or mammalian cells, as exemplified by cancer cells, lymphocytes that cause or aggravate an autoimmune disease, or senescent cells. The method also includes chemical modifications designed to increase that stability and therapeutic lifetime of selenium-carrier compounds when administered in vivo, either intravenously, inhalation, or other routes of administration. The method also teaches novel selenopeptide design features that increase its effect, such as proximity to a cell membrane or viral envelope.

For the anti-viral peptides, the organometallic compounds may be delivered, e.g., intranasally, orally (solid, liquid, or gel), intramuscularly, or intravascularly. For the anti-microbial peptides, the organometallic compounds may be delivered, e.g., orally (solid, liquid, or gel), in a cream, an eye drop, an intramuscular injection, or as an intravascular injection, depending on the target location of the infection. For the anti-cancer peptides, the organometallic compounds will find particular uses in the form of intramuscular or intravascular (solid, liquid, or gel), depending on the cancer. Of course, the route of administration can be determined by the skilled artisan to maximize the effectiveness of the dose.

The invention also introduces an entirely novel use of organometallic compounds to further increase the specific localization and effect of superoxide or perhydroxyl radicals through their conversion to the much more reactive hydroxyl radical. Such compounds may be organometallic peptide conjugates that bind to nearby sites on the surface of a viral or cellular pathogen. These novel compounds are sufficiently close to the selenium atom to efficiently carry out Fenton-like chemical reactions on selenium-generated superoxide radicals. For example, these can be nucleic acid-intercalating organometallic compounds. Cell-impermeable organometallic compounds can target hydroxyl radical damage to the genetic material of protein capsid-type viruses. Membrane-permeable intercalating compounds can target damage to nucleic acids of a wider range of viral and cellular pathogens.

Structure and basic properties of the selenium-carrier conjugate. The present invention includes methods for designing and using an optimal selenium-carrier conjugate of the form R—SeSe—R and R—SeH, wherein R is an organic compound consisting of a small organic molecule linked to a protein, synthetic peptide, chemically modified peptide or peptidomimetic compound that binds to virus-encoded surface components of a virus or virus-infected cell. The design process first produces a virus-binding protein or short peptide candidate by a genetic display technology and/or computational modeling. This protein or peptide is then further optimized by chemical additions or structurally analogous substitutions that improve the specificity of target binding and slow degradation to enhance its lifetime in or on the body. The inactive form of the conjugate, R—Se—Se—R is designed to be stable at ambient temperatures. When administered in vivo, such as intravenously, inhalation or other routes of administration, it reacts with bodily fluids or secretions containing glutathione and other reducing agents to become the active form R—Se—H. The reduced selenium acts catalytically to convert oxygen and reduced glutathione to short-lived superoxide radical (O₂).

Addition of a proton to superoxide near a membrane or other polyanionic surface produces perhydroxyl (hydroperoxyl), a more chemically reactive radical. A biologically very important chemical property of superoxide radical that has not been presented in the prior art of selenopeptides is its protonation to form the more reactive perhydroxyl radical. This reaction becomes favorable in the more acidic microenvironment that is found at the polyanionic surface of a cell plasma membrane or viral envelope. The uncharged perhydroxyl radical can then enter the lipid bilayer, causing lipid peroxidation, resulting in damage to unsaturated lipid components of the cell membrane or viral envelope. It can also enter the center of large, globular proteins found in membranes and in viruses, where it can initiate oxidative damage of the protein.

Selenopeptides can be designed to be resistant to superoxide. One advantage of a small, water soluble peptide as a selenium carrier is that it is relatively unaffected by the superoxide anions generated by the attached selenium atom. Superoxide is a very weakly reactive radical that diffuses rapidly from its source and interacts primarily with itself and other radicals, which tend to be present at very low concentrations. Avoiding internal cysteines in the peptide ensures less chemical interaction between the peptide, selenium atom and superoxide radical. Once the superoxide or perhydroxyl enters a cell, its participation in Fenton-like chemical reactions with naturally occurring iron or other transition metals produces extremely reactive hydroxyl radicals and other reactive oxygen species (ROS). Transition metals are found in the active sites of many essential cellular enzymes. Thus, the superoxide-derived hydroxyl radicals can locally disable these enzymes through chemical modification of the polypeptide chain and other components.

Distance between the selenium atom and the polyanionic cell membrane or other polyanionic surface is important. A novel feature of the method is that it considers the interaction of superoxide radical with polyanionic surfaces, especially membranes, which depress the local pH and greatly enhance protonation of superoxide to generate perhydroxyl radical. The distance between a single selenium atom and the lipid bilayer affects its ability to cause localized membrane damage. The maximum local exposure of the membrane to superoxide from the selenium atom is roughly proportional to the inverse square of the distance. This distance is important at the nanometer and sub-nanometer scale. In the case of a large transmembrane cellular or viral protein, a selenopeptide binding near the distal tip of the externally accessible portion of this protein would be significantly less effective than a peptide binding at another external site closer to the transmembrane domain.

Extremely tight binding to a molecular target is not required or desired. Another important novel design feature for a therapeutic seleno-peptide is that it does not need to bind extremely tightly to its target to be optimally effective at targeting superoxide production in order to kill cells, such as virally infected cells, and inactivate viruses. For example, this is in contrast to the use of peptides or larger proteins that are designed to block binding of a virus to its cell surface receptor. Such peptides need to bind at a specific site where virus and receptor interact. Binding of a small peptide to the virus also needs to be at least as tight as virus binding to its receptor. This is not easily achievable when normal binding of a virus and its receptor generally involve a wider area of protein-protein or protein-carbohydrate interaction. Mutations that cause small changes in amino acids of the virally-encoded receptor-binding domain may weaken its interaction with the peptide, or strengthen interaction of the virus with its natural receptor. Such a mutation in the virus genome could make the new viral strain resistant to a peptide blocking therapy. In contrast, the selenium-peptidomimetic compound only needs to be bound to the virus or cellular target long enough to inactivate it by localized production of superoxide. Similar outcomes hold for physiologically functional target molecules on cellular pathogens or abnormal human cells.

It has been found that selenopeptides are much more effective at inhibiting virus infection than blocking peptides. In terms of the stoichiometry of interaction, the selenopeptide does not need to saturate most of the viral surface proteins, as required of a blocking peptide. The present invention was successfully used to demonstrate that a viral CD4-derived peptide as previously designed to competitively block HIV virus infection can be used with the present invention. The effect of this peptide on interactions between virus gp 120 and CD4, its receptor, was only observed at a relatively high concentration of 457 micrograms per ml of synthetic peptide. The same peptide conjugated to selenium gave over 99% inhibition of cell infection at 0.5 micrograms per ml, and over 50% inhibition at 0.006 micrograms per ml.

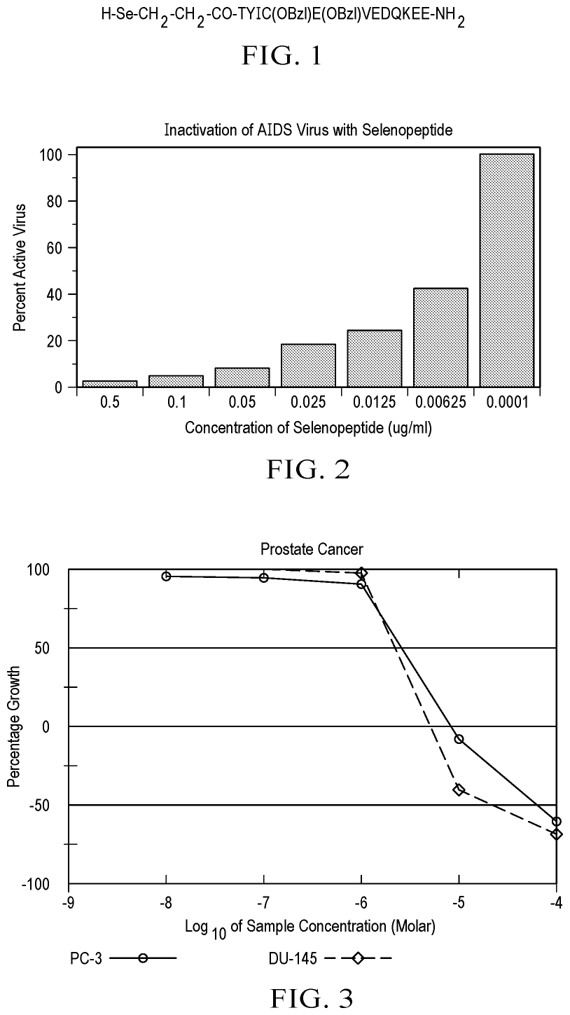

FIG. 1 shows the sequence of the peptide Thr Tyr Ile Cys Glu Val Glu Asp Gln Lys Glu Glu (SEQ ID NO: 1) mixed with the viral isolate and allowed to stand for one hour in the presence of glutathione (250 uM). The viral solution was then diluted 100-fold and used in a cell infectivity assay to measure active virus.

FIG. 2 is a graph that shows effectiveness at a very low concentration of seleno-peptide (0.1 ug/ml), which resulted in 95% inactivation of HIV-1 (this was measured by the ability of the infected cells to produce the virus). A control solution of peptide that did not contain selenium had no effect. This shows that a specific selenopeptide can bind to HIV and inactivate it is one hour at a concentration of peptide of 0.5 ug/ml.

Catalytic function of a selenopeptide. Another feature that enhances the effectiveness of an anti-viral selenopeptide is that it functions catalytically. It can therefore be effective at an intermediate level of virus affinity, one where the anti-viral selenopeptide can detach and re-attach multiple times. The off-rate of this binding equilibrium is optimized to allow the selenium conjugate to detach and re-attach to another virion or cell, thereby enabling the catalytic inactivation of multiple virions per molecule of selenium conjugate. Again, having reduced affinity is an advantage of the selenopeptides of the present invention. This provides an anti-viral therapeutic working at lower dosages than either anti-virion antibodies or peptides designed to simply block virus-receptor binding by competitive inhibition.

Cell killing by targeted selenopeptides. The additional ability of targeted selenopeptides to kill infected mammalian cells that display virus envelope membrane proteins on their surface suppresses the production of new virus by infected cells. This is a modality that is not available for peptides, proteins or antibodies that work by blocking virus entry. By killing cells displaying viral coat proteins, the selenopeptide mimics and complement the natural function of cytolytic T lymphocytes, which are specialized to identify and destroy virus-producing/infected cells. A similar cell killing function applies to selenopeptides that target proteins or other molecules specifically located on the external surfaces of cellular pathogens, such as bacteria, fungi, protozoans. Formally, these cellular pathogen countermeasures can also be used to target human or mammalian cancer cells that exhibit unique or rare cell surface molecules.

Limitations of selenium-generated superoxide. Cells have a complex internal metabolism and a large DNA genome that provides many more targets than viruses. However, cells also have an effective internal machinery for neutralizing small amounts of superoxide, which is normally produced endogenously as a byproduct of several metabolic processes. This cellular machinery includes abundant superoxide dismutase enzymes that convert superoxide to less reactive hydrogen peroxide and oxygen. Cells also produce catalases that finally convert hydrogen peroxide to oxygen and water. Some pathogenic bacteria have even enhanced these defenses to superoxide in order to evade immune lymphocytes that utilize superoxide, hydrogen peroxide or other reactive oxygen species to kill invasive bacteria.

In mammalian viral pathogens, many produce virions that are not protected by a membrane envelope, and therefore may not be as efficiently inactivated through the conversion of localized superoxide to more reactive perhydroxyl radicals by the polyanionic lipid bilayer. Pathogenic human and mammalian viruses are found among a number of virus groups which have protein capsids and lack envelopes:

Picornaviridae, a family of + strand RNA viruses that include poliovirus, enterovirus, rhinovirus, and hepatitis A virus.

Flaviviridae, a family of + strand RNA viruses that include the mosquito-borne Flavivirus genus that includes the Yellow fever virus, West Nile virus, Dengue virus, and Zika virus. It also includes the Hepacivirus genus, of which includes the Hepatitis C virus, as well as the Pegivirus genus, which contains species that can cause of persistent hepatitis.

Reoviridae, a family of double stranded RNA viruses that includes the genus Rotavirus, which contains viruses causes gastrointestinal infections.

Papovaviridae, a family of double stranded DNA viruses that contains the genus Papillomavirus, which contains species that can cause warts and others that can cause human cervical cancer.

Adenoviridae, a family of double stranded DNA viruses that cause respiratory and gastrointestinal infections.

Use of a second organometallic compound as a Fenton complex to boost magnitude and selectivity of selenopeptide action. To address these potential shortcomings of a selenium-carrier strategy, the present invention provides a method for greatly boosting the magnitude and selectivity of molecular damage to the pathogen. This involves placing iron or transition metal atoms close enough to the selenium atom that they can carry out a Fenton-like reaction that converts superoxide to the electrically neutral and extremely reactive hydroxyl radical. Hydroxyl radicals are typically short-lived and more highly reactive than either the superoxide or perhydroxyl radical. Hydroxyl radicals react with polypeptides, unsaturated lipids, as well as RNA and DNA. They are capable of causing major damage to cell membranes, proteins and nucleic acids.

Fenton complex-1: Peptide-derived carrier for iron or transition metal. One approach is to design an iron carrier molecule using the same general strategy employed for the selenium carrier. For example, in the case where the surface target is a multimeric protein, targeting iron using exactly the same carrier peptide would often place an iron atom on a neighboring subunit of the same multimeric protein. If the carrier design process yielded several peptides, each binding to independent sites on the same protein subunit, there would be an advantage to using a different targeting peptide as the iron carrier. This could add further specificity to localized radical production, helping to minimize any off-target effects. Because it is intrinsically less toxic, in a clinical setting an iron conjugate might be administered at the same or higher dosage than the selenium conjugate.

Fenton complex-2: DNA and RNA intercalating small organometallics. A different approach is to broadly target viral or cellular nucleic acid via an intercalating organo-ferric or organo-metallic compound. It may be helpful to design such compounds, although some intercalating organo-metallic compounds suitable for this purpose, such as cis-platin, are already in use as anti-cancer agents. Existing intercalating organometallics are sufficiently hydrophobic that they can enter cells and enveloped viruses with reasonable efficiency, and then bind to DNA and RNA. For targeting protein capsid-type viruses it may be optimal to design more water soluble oragnometallics that are generally excluded from cells but can readily diffuse into the core of the virion containing RNA or DNA.

Fenton complex-3: pathogen binding ferritin. In this embodiment, the Fenton complex's pathogen-binding peptide or peptidomimetic is cross-linked to a purified ferritin, and then administered in vivo. Alternatively, the Fenton complex's pathogen-binding peptide or peptidomimetic would be extended with a ferritin-binding peptide motif. This would independently recruit naturally present serum or interstitial ferritin to the surface of the pathogenic virus or cell. Proximity of the iron atoms carried by transferrin would provide a local Fenton-like conversion of superoxide to hydroxyl radical. Analogously to natural iron processes, superoxide radicals would be transformed into hydroxyl radicals that would damage the protein shell of the ferritin complex, allowing leakage of its contents. Gradual release of the many (up to 4500) iron atoms contained within. These released iron molecules would convert superoxide hydroxyl radicals and cause localized damage. The iron in ferritin is stored as a relatively insoluble iron, but would undergo chemical transformation and be progressively released from the damaged ferritin shell in more soluble form. This increase in soluble extracellular iron would boost hydroxyl radical production by Fenton reactions in the local environment of targeted cells. Once the pathogens are destroyed, the free iron would eventually be sequestered and cleared by transferrins in the serum.

Tolerance of selenopeptides and organometallic Fenton complexes. Selenium is an essential human micronutrient, with well characterized levels of toxicity at high concentrations. Selenium released by complete metabolism of anticipated doses of selenopeptides would be well below the amount that is normally tolerated and excreted by the body. Fenton complexes would typically contain iron, which is readily handled by the body in larger quantities. Other transition metals such as copper or nickel have higher toxicity. Like selenopeptides, they would be used in small quantities that are well below toxic limits. Biological intolerance or immune reactions to the peptide or peptidomimetic portion of the conjugate would be the remaining concern, however, small, soluble peptides have the advantage that they are less likely to generate an immune response than large globular proteins. This concern is readily addressed by animal and human testing.

Application to the treatment of cancer or other diseases by targeted cell ablation. The combined use of a selenopeptide with a Fenton complex provides the ability to target the combination of two proteins on the surface of a cell. In the right circumstances, this could provide an improved way to ablate a particular normal, infected, and/or aberrant cell type. Antibody targeted selenium has been proposed as a way of killing cancer cells that carry the recognized antigen. Requirement for the cognition of a second cell surface protein by peptide would add further selectiveness to this process. This selectivity could make the method also useful for ablating abnormally functioning cell types. One example would be autoimmune diseases, where a specific class of lymphocyte or other immune cell could be selectively decreased in number or eliminated.

In one example, the present invention is a method of making a selenium-carrier conjugate for in vivo administration comprising: covalently attaching a selenium compound that reacts in vivo with naturally occurring reduced thiols and oxygen to produce superoxide and perhydroxyl radicals, to a synthetic targeting carrier to form the selenium-carrier conjugate, wherein the selenium-carrier conjugate may be stabilized against enzymatic degradation, and clearance from the body. The synthetic targeting carrier binds with high specificity to an external domain of a targeted protein or membrane protein on the surface of an animal virus, cellular microbe, or mammalian cell wherein the selenium-carrier conjugate produces superoxide radicals and their protonated derivative, the perhydroxyl radical, which locally damages lipids in viral envelopes or proteins in viral capsids to inactivate animal viruses or kill one or more bacteria, fungi, protozoans, cancerous or otherwise disease-causing mammalian cells and selectively kills infected cells infected with the one or more membrane-enveloped viruses.

The synthetic targeting carrier for selenium is a chemically modified peptide or peptidomimetic molecule that specifically binds to the targeted protein, membrane protein, carbohydrate or other biological macromolecular assembly. For example, a peptide precursor of the synthetic peptidomimetic targeting carrier is designed by expressing and displaying a library of peptides having different amino acid sequence permutations, then selecting phage expressing peptides with the desired properties. One embodiment of this is to express the permuted amino acid peptide sequence within the polypeptide chain or the surface protein of a bacteriophage protein and selecting the bacteriophage that bind to the targeted protein on the surface of one or more viruses or cells. The starting point of the selection process may be either completely random permutations of an amino acid peptide sequence or a partially random amino acid peptide sequence permutation encoded in the genome of and displayed as part of a protein of the corresponding capsid surface of a bacteriophage. Selections are made by binding assays using a very large collection of such bacteriophage known as a library. The peptide precursor sequences are selected to bind at positions within a few nanometers of a membrane of the one or more membrane-enveloped viruses or cells infected with the one or more membrane-enveloped viruses to enhance the efficiency of virus inactivation or infected cell killing by their selenium-carrier conjugates.

The initial binding peptides can be identified by, for example, bacteriophage display or another molecular display method. Next, the peptides selected can be further modified by at least one of, for example, extending the N-terminus and C-terminus by adding chemical groups that hinder the action of terminal peptidases; coupling carbohydrate polymers that increase solubility and extend the lifetime of the selenium-carrier complex: changing the amino acid sequence, chemically modifying the amino acids, substituting one or more peptide linkages, or substituting the peptide chain with one or more D-amino acids, wherein the modifications enhance binding affinity of the modified peptide precursor to the target and increase its resistance to proteolysis in vivo. Such modifications will likely increase clinical effectiveness of the selenium-carrier conjugate.

The selenium-carrier conjugate or selenium-targeting carrier is designed to function catalytically, so that its binding and dissociation rate constants are such that it only attaches transiently to the target site, such that the seleno-conjugate detaches and reattaches multiple times, allowing the selenium-carrier conjugate to destroy multiple viral or cellular targets, either in vitro or in vivo. Examples of membrane-enveloped viruses include, e.g., a coronavirus, influenza virus, human immunodeficiency virus (HIV), respiratory syncytial virus (RSV), herpesvirus or other membrane-enveloped human or animal virus. Also included as targeted virus particles are protein-encapsidated non-envelope viruses. These include viruses of the following genera and species: Adenovirus, Poliovirus, Enterovirus, Rhinovirus, Hepatitis A virus, Yellow fever virus, West Nile virus, Dengue virus, Zika virus, Hepatitis C virus, Rotavirus, Papillomavirus. For membrane-enveloped viruses the selenium-carrier conjugates can be selected to selectively kill cells productively infected by binding to the excess virally-encoded envelope proteins that frequently appear on the plasma membrane surface of cells infected with membrane-enveloped viruses during their replication in that cell. Killing virus-infected cells with the selenium-targeting carrier is an effective added countermeasure that can greatly decrease the production of new virus particles. Selective binding of the selenium-targeting carrier and the short range of perhydroxyl radical action will spare uninfected neighboring cells.

The selenium is specifically targeted to the exposed surfaces of cells for the purpose of killing these cells. These cells may either be cellular pathogens such as bacteria, fungi, or protozoans, or abnormal human or mammalian cells such as cancers or otherwise dysfunctional cells. Typically, the selenium-carrier conjugate is stored as a relatively stable and inactive diselenide dimer of the form R—Se—Se—R or R—Se—Se—R′, at a suitable ambient temperature that could range from −20C to 40C. The selenium-carrier conjugate and/or the inactive diselenide dimer can administered intravenously, orally, topically, nasally, or through pulmonary administration by inhalation of an aqueous mist or dry powder to epithelia of the upper and lower respiratory tract, wherein the inactive diselenide dimer is converted to superoxide-generating R—Se—H monomers by in situ glutathione and other reducing compounds.

A human or animal patient can be treated after viral exposure or during an active viral infection by providing a synthetic targeting carrier covalently attached to a selenium compound, with intravenous, nasal, oral, topical or pulmonary administration of said carrier; then reacting in vivo with naturally occurring thiols and oxygen to catalytically generate many short-lived superoxide and perhydroxyl radicals. The selenium-carrier conjugate binds with high specificity to the external domain of a targeted membrane protein on the surface of a membrane-enveloped animal virus or the surface plasma membrane of a virus-infected cell. The attached selenium-carrier conjugate generates superoxide radicals that transform into perhydroxyl radicals at the acidic membrane interface or other polyanionic environment within protein-encapsidated viruses. The perhydroxyl radicals cause sufficient oxidative damage to membrane lipids of the virus to render its protective membrane permeable, and to further inactivate the targeted virus by damage to its proteins and enclosed RNA or DNA nucleic acid genome. Similarly, the superoxide and highly reactive perhydroxyl radicals generated at the plasma membrane of virus-infected cells can cause selective killing of these cells. A patient is provided with a sufficient amount of this selenium-carrier conjugate, as formulated for in vivo administration, in order to effectively reduce the levels of active virus and viral infection.

The synthetic targeting carrier is a peptidomimetic molecule that specifically binds to a targeted viral envelope membrane protein, which may also be present in the plasma membrane on the surface of infected cells. A peptide precursor of the peptidomimetic synthetic targeting carrier is made by: expressing and displaying a library of peptides having different amino acid sequence permutations on the surface of a bacteriophage, and selecting the bacteriophages that bind to the targeted transmembrane protein on the surface of the one or more membrane-enveloped viruses.

The synthetic targeting carrier can be further improved by modifying a selected peptide precursor by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo. Such modifications are of advantage for in vivo administration, especially for oral administration in treating gastrointestinal viruses, where unmodified peptides are unlikely to survive transit through the upper gut before reaching sites of infection in the small and large intestine. Such modifications increase chemical stability of an inactive diselenide form of the selenium-carrier conjugate, such that it can be stored at a wide range of ambient temperatures prior to administration. After the inactive R—Se—Se—R or R—Se—Se—R′ diselenide dimer is administered by intravenous, oral topical, nasal or pulmonary routes, it is converted to superoxide-generating R—Se—H monomers in situ by interaction with reduced glutathione and molecular oxygen. The one or more membrane-enveloped viruses is a coronavirus, influenza virus, human immuno-deficiency virus (HIV), respiratory syncytial virus (RSV), or other membrane-enveloped virus. In other examples, the targeted virus particles are protein encapsidated non-envelope animal viruses such as the following genera and species: Adenovirus, Poliovirus, Enterovirus, Rhinovirus, Hepatitis A virus, Yellow fever virus, West Nile virus, Dengue virus, Zika virus, Hepatitis C virus, Rotavirus, Papillomavirus. For such non-enveloped viruses, production of the effective perhydroxyl radical is entirely dependent on the presence of protein and nucleic acid polyanions. Lower levels of superoxide conversion to perhydroxyl are expected for targeted protein capsid, non-enveloped viruses. For such non-enveloped viruses, proximity of the targeted selenium-carrier conjugate to the virus will be of greater importance. It is anticipated that higher dosages of the selenium-carrier conjugate or enhanced methods of administration may be required, or that the Fenton complex method introduced in claim 28 may be necessary to achieve sufficient reactive radical production for therapeutic effect. Achieving a high efficiency of viral particle inactivation may be of special importance for non-enveloped viruses due to the fact that their protein components are generally not displayed on the surface of the infected cell plasma membrane during viral replication, but are only released after rupture and death of the infected cell. Cells infected by non-enveloped viruses are not as readily targeted by selenium-carrier conjugates.

Another example of the present invention is a method of treating a human or animal patient infected with cellular microbial pathogens such as bacteria, fungi or protozoans by providing or making a synthetic targeting carrier covalently attached to a selenium compound, with intravenous, nasal, oral, topical or pulmonary administration of said carrier; then reacting in vivo with naturally occurring thiols and oxygen to catalytically generate many short-lived superoxide and perhydroxyl radicals. Again, this selenium-carrier conjugate binds with high specificity to the external domain of a targeted cell membrane protein on the surface of an actively growing and proliferating cell, or a relatively inactive cyst-like form of the cellular microbe. The attached selenium-carrier conjugate generates superoxide radicals that transform into perhydroxyl radicals at the acidic plasma membrane interface. The perhydroxyl radicals cause sufficient oxidative damage to plasma membrane lipids to render the cell permeable and cause lysis, and sufficiently damage its DNA through mutations or chromosomal breaks to prevent significant replication of cells or cyst. The method also includes providing a patient with a sufficient amount of this selenium-carrier conjugate, as formulated for in vivo administration, in order to effectively reduce the levels of actively proliferating pathogen and, where relevant, to prevent inactive cysts or spores from producing actively proliferating pathogens.

The synthetic targeting carrier is a peptido-mimetic molecule that specifically binds to the targeted pathogen-encoded cellular plasma membrane protein or a specific protein located on the surface of a spore or cyst form of the pathogen. In addition, bacteria, fungal cells and spores, as well as protozoal cysts often have distinctive cell walls constructed of carbohydrate and carbohydrate-peptide polymers that are potential specific binding targets for selenium-carrier conjugates. A peptide precursor of the peptidomimetic synthetic targeting carrier is made by: expressing and displaying a library of peptides having different amino acid sequence permutations on the surface of a bacteriophage, and selecting the bacteriophages that bind to the targeted transmembrane protein on the surface of the one or more membrane-enveloped viruses.

A selected peptide precursor can be further modified by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo. Such modifications are advantageous for in vivo administration, especially for oral administration in treating gastrointestinal infections, where unmodified peptides are unlikely to survive transit through the upper gut before reaching sites of infection in the small and large intestine. Such modifications may increase chemical stability of an inactive diselenide form of the selenium-carrier conjugate, such that it can be stored at a wide range of ambient temperatures prior to administration. After the inactive R—Se—Se—R or R—Se—Se—R′ diselenide dimer is administered by intravenous, oral topical, nasal or pulmonary routes, it is converted to superoxide-generating R—Se—H monomers in situ by interaction with reduced glutathione and molecular oxygen.

For treatment of bacterial infections, the present invention can be designed to kill or permanently inactivate actively growing, inactive, latent or spore-like forms of specific bacteria. The inactive but viable forms of bacteria are difficult to treat with current antibiotic drugs, and recovery usually depends on the patient's immune system to finally clear the infection. Other bacterial species and genera, such as Mycobacterium tuberculosis. Mycoplasma, and Chlamydia, enter and proliferate within cells, out of reach of the immune system, and may remain in latent form within these cells. In these cases, the selenium carrier-conjugate is designed to target, for example, the path of the bacterium into the cell, whether by penetrating into the cytoplasm (Mycoplasma) or endocytosis and entry into the microbe-modified endocytic vesicle within the cell (M, tuberculosis and Chlamydia). This occurs by binding the selenium carrier-conjugate tightly to bacteria before they enter a cell, or by designing a peptidomimetic that also directs endocytosis of the selenium carrier-conjugate. The latter might be especially useful in addressing the latent state of Mycobacterium tuberculosis infection.

Treatment of fungal infections with selenium-carrier conjugates can be used for a number of opportunistic pathogens in genera, such as Histoplasma, Pneumocystis, Coccidiomyces, Candida. The selenium carrier-conjugate can target both actively growing fungi outside and inside host cells of the patient, as well as inactive spore forms that may be responsible for persistence and transmission of the infection. Fungal infections are often most serious in patients who are immunosuppressed, either because of a disease that weakens their immune system, or because they are under treatment for cancer or other diseases. Since selenium-carrier conjugates do not depend on function of the immune system, they provide a special value in the treatment of these patients.

Treatment of protozoal infections with selenium-carrier conjugates works by killing protozoal pathogens. Like the fungi, they are microbial eukaryotes with complex life cycles and multiple cell types. Unlike the plant-like fungi, they are motile, and can actively move into different environments. Plasmodium falciparum, the pathogen that causes malaria, has different stages of its life cycle where it can live in the insect gut, human liver cells, inside human red blood cells, with intermediate steps when it is present and accessible in the serum. It is adept at evading immune responses by growing inside cells and changing its surface antigens. In this case, the targeted plasma membrane proteins must be those expressed when the organism is free in the blood, and more specifically those protein motifs that remain constant during its switches in expression to different immunologically active surface glycoporoteins. The selenium-carrier conjugates will most often be administered intravenously. A similar approach would apply for Trypanosoma cruzi, the cause of Chagas disease. A very different example is Entamoeba histolytica, which causes severe gastrointestinal disease. In this case the peptidomimetic selenium-carrier conjugates is made insensitive to proteases, so that they could be administered orally and enter the lumen of the intestinal tract. Entamoeba histolytica produces a spore-like cyst that might need to be targeted independently.

The present invention can also be used in a method of treating a human or animal patient with a cancer or other disorder caused by abnormal cells: comprising a synthetic targeting carrier covalently attached to a selenium compound, with intravenous, nasal, oral or pulmonary administration of said carrier; then reacting in vivo with naturally occurring thiols and oxygen to catalytically generate many short-lived superoxide radicals. The selenium-carrier conjugate binds with high specificity to the external domain of a targeted membrane protein on the surface of a cancer cell or a specific abnormal cell type, selenium carrier-conjugate attached selenium-carrier conjugate generates superoxide radicals that transform into perhydroxyl radicals at the acidic plasma membrane interface, selenium carrier-conjugate perhydroxyl radicals cause sufficient oxidative damage to membrane lipids and proteins to lyse the cell, or sufficient damage to nuclear DNA permeable to disable continued proliferation or function of the cell, selenium carrier-conjugate method provides a patient with a sufficient amount of this selenium-carrier conjugate, as formulated for in vivo administration, in order to kill or block proliferation of cancer cells. In the case of abnormal immune cells underlying autoimmune disorders such as multiple sclerosis, lupus or Graves' disease, the method provides a feasible method for developing therapies that specifically reduce or remove dysfunctional cells. Since the targets are normal proteins expressed in abnormal locations, specificity of effect is crucial. For this reason, use of the Fenton complex (see discussion below) may be advantageous or essential. It increases specificity by requiring cells to express two different cell surface targets.

In this example, the synthetic targeting carrier is a peptido-mimetic molecule that specifically binds to the targeted cellular plasma membrane protein specific to a cancer cell or another abnormal, disease-causing cell of the patient. Wherein a peptide precursor of the peptidomimetic synthetic targeting carrier is made by: expressing and displaying a library of peptides having different amino acid sequence permutations on the surface of a bacteriophage, and selecting the bacteriophages that bind to the targeted transmembrane protein on the surface of the one or more membrane-enveloped viruses.

A selected peptide precursor can be modified by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo. Such modifications are of special advantage for in vivo administration, especially for oral administration in treating gastrointestinal cancers, where unmodified peptides will not survive transit through the upper gut before reaching the targeted cells. Such modifications may increase chemical stability of an inactive diselenide form of the selenium-carrier conjugate, such that it can be stored at a wide range of ambient temperatures prior to administration. After the inactive R—Se—Se—R or R—Se—Se—R′ diselenide dimer is administered by intravenous, oral topical, nasal or pulmonary routes, it is converted to superoxide-generating R—Se—H monomers in situ by interaction with reduced glutathione and molecular oxygen.

The selenium carrier-conjugate of the present invention have the ability to selectively ablate cells by a mechanism that does not depend on radioactive isotopes or the immune system opens up new approaches for treating cancers, autoimmune disorders and possibly some neurological conditions. In cases where stem cell-based cell replacement in highly specialized neural organs such the retina, this method could prove useful in removing specific defective cell types in order to create niches that can be occupied by their replacements.

Thus, the selenium carrier-conjugates of the present invention can be designed to have antiviral antibacterial, antifungal, anti-protozoal, anticancer or applications involving removal of abnormal human or animal cells, where increased effect, range and specificity of action of a selenium-carrier complex are considered advantageous, can be enhanced through the addition of a second targeted compound named a Fenton complex, to be administered in combination with the chosen selenium-carrier complex.

As used herein, the term “Fenton complex” refers to an organometallic compound or conjugate containing one or more atoms of iron, copper or other transition metals that can catalyze the conversion of superoxide, perhydroxyl radical or hydrogen peroxide into other compounds, yielding products that may include hydroxyl radicals and other reactive oxygen species. The hydroxyl radicals are generated locally by the Fenton complex metal atoms, and have their greatest effect very close to these metal atoms because of their highly reactive nature, which also enables them to chemically modify a wide range of molecules, including membrane lipids, polypeptides, RNA, DNA, carbohydrates and other biological molecules.

The present invention includes a combination of the selenium-carrier and Fenton complex that may either be simultaneous, or sequential administration in vivo, whichever order is found to be advantageous. In addition, the dosages and molar ratios of the selenium-carrier and chosen Fenton complex may require experimental optimization. The organo-metallic compound can includes an iron, copper or other transition metal covalently attached to, tightly chelated by, coordinated with or enclosed by a small organic molecular framework. These small organic molecular frameworks may include synthetic metal-coordinating compounds, or naturally occurring siderophores coupled to the peptide or peptidomimetic targeting molecule. These frameworks would be loaded with the appropriate tightly bound or encaged metal ion prior to final purification, storage and in vivo administration. The organo-metallic can be chemically modified or extended to produce an organic molecule that can bind to either RNA, DNA or both by its ability to intercalate between the bases of the polynucleotide. For example, RNA or DNA intercalating organo-metallic compounds can be designed or selected to be membrane-permeable, and able to combine with the RNA or DNA genetic material of enveloped viruses, thereby targeting the production of hydroxyl radical and its damaging inactivating and mutagenic effects on the membrane-enveloped virus particles.

Alternatively, the RNA or DNA intercalating organo-metallics can be designed to be membrane-impermeable, such that the Fenton complex will preferentially bind to the genetic material of protein-encapsidated non-enveloped virus particles, thereby restricting the production of hydroxyl radical and its highly damaging effects to genetic material of these types of viruses. This type of Fenton complex would provide an additional level of control to further minimize possible off-target free radical damage to the genome or intracellular components of human or mammalian cells. The organo-metallic compound can be chemically linked to a peptide or peptidomimetic compound designed as previously described such that it will bind with high selectivity to the same protein that is the binding target of the peptide used to target selenium to the surface of the virus, as described in claim 1. The peptide or peptidomimetic of the Fenton complex can be identical to the peptide or peptidomimetic synthetic carrier that is conjugated to the selenium compound.

For example, where the viral protein or other molecule that selectively binds to this carrier exists as a dimer, trimer, or higher level multimer, a significant fraction of the superoxide-generating selenium atoms are positioned within nanometers of Fenton complex metal atoms, which will convert these into highly reactive hydroxyl radicals. The peptide or peptidomimetic carrier for the Fenton complex may include different chemical composition and have a different viral binding site specificity from the peptide or peptidomimetic synthetic carrier that is conjugated to the selenium compound described above. In cases where the carrier selectively binds to a different and non-competing site on the same target viral protein molecule, superoxides will be generated within nanometers of the Fenton complex metal atoms, which will convert these into highly reactive hydroxyl radicals.

In cases where the Fenton complex and selenium-conjugate selectively bind to two different proteins that are localized to the same plasma membrane domain of a virus-infected cell, same viral envelope or same viral capsid. Fenton complex metal atoms will convert a fraction of the selenium-generated superoxide into highly reactive hydroxyl radicals. For in vivo administration it may be advantageous to use higher dosages of a relatively lower toxicity iron-based Fenton complex in conjunction with a smaller dosage of the potentially more toxic selenium conjugate. In cases where the target is a virus defined by the combination of two independent peptide binding sites on one large surface protein, or two separate but intermixed proteins on the surface of the viral membrane envelope or protein capsid, targeted viral inactivation with two different carriers has the potential for a greater degree of selectivity than methods based on a single binding site.

A Fenton complex can be an iron-filled ferritin complex containing roughly 4500 iron atoms brought into the proximity of the active selenium-conjugate by a targeting peptide, peptidomimetic, protein or antibody. By bringing ferritin close to the targeted selenium, superoxide radicals produced by selenium will diffuse into the large ferritin-iron complex, where the iron would catalytically convert them to highly reactive hydroxyl radicals. These hydroxyl radicals would eventually damage the ferritin shell, leading to local release of iron in the vicinity of the selenium-targeted virus or virus-infected cells. The local presence of many soluble iron atoms would greatly enhance the local effectiveness of the targeted selenium. This enhanced Fenton or Fenton-like effect would be transient, since the released iron would eventually be sequestered and removed by transferrin normally present in serum and in interstitial spaces around tissues. However, it would significantly enhance viral inactivation and infected cell killing by the targeted selenium atoms in regions with active viral infection. The Fenton complex can also be constructed by using a chemical linker to covalently cross-link a single ferritin complex to each targeting peptide, peptidomimetic, protein or antibody. For example, its possible to use a purified human ferritin-iron complex, either from organs, cultured human cells, or by expression of human recombinant heavy and light chain proteins followed by self-assembly of the iron-ferritin complex. Alternatively, ferritin-iron complex could be obtained from an animal source that is, for practical purposes, immunologically compatible.

In another example, the virus and cell-targeting carrier of the Fenton complex peptide, protein, antibody or peptidomimetic would be extended by the addition of an antibody or designed peptide that would confer the additional ability to bind selectively to human ferritin protein. After in vivo intravenous administration, this would allow the targeting carrier to first bind to a circulating ferritin-iron complex, which is found normally in blood at reasonable abundance. The targeting carrier would then bind virus particles or virus-infected cells, thereby bringing an iron-ferritin complex near some of the targeted selenium-carrier molecules.

For specific applications designed to kill cellular pathogens including bacteria, fungi and protozoans (where increased effect, range and specificity of action of a selenium-carrier complex are considered advantageous), the selenium-carrier conjugate can be enhanced through the addition of a second targeted compound named a Fenton complex, to be administered in combination with the chosen selenium-carrier complex. Again, the hydroxyl radicals generated locally by the Fenton complex metal atoms, have their greatest effect very close to these metal atoms because of their highly reactive nature, which also enables them to chemically modify a wide range of molecules, including membrane lipids, polypeptides. RNA. DNA, carbohydrates and other biological molecules. Thus, a combination of the selenium-carrier and Fenton complex, may either be simultaneous, or sequential administration in vivo, whichever order is found to be advantageous. In addition, the dosages and molar ratios of the selenium-carrier and chosen Fenton complex may require experimental optimization. In this example, organo-metallic compound may be iron, copper or other transition metal covalently attached to, tightly chelated by, coordinated with or enclosed by a small organic molecular framework. These frameworks may include synthetic metal coordinating compounds, or naturally occurring siderophores coupled to the peptide or peptidomimetic targeting molecule. These frameworks would be loaded with the appropriate tightly bound or encaged metal ion prior to final purification, storage and in vivo administration. As with previous examples, the organo-metallic compound can be chemically modified or extended to produce an organic molecule that can bind to either RNA. DNA or both by its ability to intercalate between the bases of the polynucleotide.

In this example, the RNA or DNA intercalating organo-metallic compound can be designed to be membrane-permeable, and able to combine with the genetic material of cells, thereby targeting the production of hydroxyl radical and its damaging effects to the genetic material of the cellular pathogen. Since the selenium atom is targeted to cellular pathogens, the production of superoxide radical is specifically targeted to these. Intercalation of the Fenton complex in DNA or RNA of the human or mammalian patient would therefore have much less effect on the patient's cells.

Alternatively, the RNA or DNA intercalating organo-metallic compound can be designed to be membrane-impermeable, such that it will preferentially bind to the genetic material of cells that either selectively import the carrier via a pathogen-specific transporter, have sustained membrane damage from superoxide produced by the targeted selenium atom, or are otherwise inherently leaky to this agent, thereby targeting the production of hydroxyl radical and its damaging effects to genetic material of the cellular pathogen. This type of Fenton complex would provide an additional level of control to avoid targeting free radical damage to genomic DNA or other intracellular components of healthy human or mammalian patient cells. Again, the organo-metallic compound can be chemically linked to a peptide or peptidomimetic compound designed as previously described such that it will bind with high selectivity to the same protein produced by the cellular pathogen that is the binding target of the peptide used to target selenium to the surface of the cell, as described in claim 1. The peptide, protein, antibody or peptidomimetic for the Fenton complex may be identical to the peptide, protein, antibody or peptidomimetic synthetic carrier that is conjugated to the selenium compound described hereinabove. In cases where the protein or other molecule that selectively binds to this carrier exists as a dimer, trimer, or higher level multimer, a significant fraction of superoxide-generating selenium atoms will be positioned within nanometers of Fenton complex metal atoms, which will convert these into highly reactive hydroxyl radicals. Alternatively, the peptide, protein, antibody or peptidomimetic of the Fenton complex, with a different binding site specificity from the peptide or peptidomimetic synthetic carrier that is conjugated to the selenium compound described above. In cases where the carrier selectively binds to a different and non-competing site on the same target protein, superoxide will be generated within nanometers of the Fenton complex metal atoms, which will convert these into highly reactive hydroxyl radicals. In cases where the Fenton complex and selenium-conjugate selectively bind to two different proteins that are localized to the same cell and plasma membrane domain. Fenton complex metal atoms will convert a fraction of the selenium-generated superoxide into highly reactive hydroxyl radicals. For in vivo administration it may be advantageous to use higher dosages of a relatively lower toxicity agent, such as the iron-based Fenton complex in conjunction with a smaller dosage of the selenium conjugate. In cases where the target is a cellular pathogen defined by the combination of two independent peptide binding sites on one large surface protein, or two separate but intermixed proteins on the surface of the pathogen's cell membrane, targeted cell killing with two differently targeted selenium and transition metal carriers, has the potential for a greater degree of selectivity than methods based on a single binding site.

Another example of a Fenton complex is an iron-filled ferritin complex containing roughly 4500 iron atoms brought into the proximity of the active selenium-conjugate by a targeting peptide, peptidomimetic, protein or antibody. The rationale is that by bringing ferritin close to the targeted selenium, superoxide radicals produced by selenium would diffuse into the large ferritin-iron complex, where the iron would catalytically convert them to highly reactive hydroxyl radicals. These hydroxyl radicals would eventually damage the ferritin shell, leading to local release of iron in the vicinity of the selenium-targeted cells. The local presence of many soluble iron atoms would greatly enhance the local effectiveness of the targeted selenium. This enhanced Fenton or Fenton-like effect would be transient, since the released iron would eventually be sequestered and removed by transferrins normally present in serum and in interstitial spaces around tissues. However, it would significantly enhance bacterial, fungal or protozoal killing in the region of infection by the targeted selenium atoms. The Fenton complex would be constructed by using a chemical linker to covalently cross-link a single ferritin complex to each targeting peptide, peptidomimetic, protein or antibody. For use in humans, a purified human ferritin-iron complex, either from organs, cultured human cells, or by expression of human recombinant heavy and light chain proteins followed by self-assembly of the iron-ferritin complex. The other animal use, the ferritin can be isolated from the target animal. Alternatively, ferritin-iron complex could be obtained from an animal source that is, for practical purposes, immunologically compatible. The cell-targeting carrier of the Fenton complex peptide, protein, antibody or peptidomimetic would be extended by the addition of an antibody or designed peptide that would confer the additional ability to bind selectively to human ferritin protein. After in vivo intravenous administration, this would allow the targeting carrier to first bind to a circulating ferritin-iron complex, which is found normally in blood at reasonable abundance.

For specific applications designed to kill cancer cells and other abnormal, dysfunctional cells in the body, where increased effect, range and specificity of action of a selenium-carrier complex are considered advantageous, can be enhanced through the addition of a second targeted compound named a Fenton complex, to be administered in combination with the chosen selenium-carrier complex. Cancer cells can often be identified by single surface proteins or combinations of cell surface proteins that are distinctive to the cancer cell. It may also be beneficial to remove abnormal cells that are pre-cancerous, senescent, inappropriately secrete signaling molecules, or are otherwise dysfunctional. One clinically important class of abnormal cells is represented by immune cells that underly the causes of autoimmune diseases and dangerous allergies. As with cancer cells, these can also bear distinctive combinations of surface proteins, and in specific instances might be effectively targeted by a combination of selenium and Fenton complex. Again, a Fenton complex, can include an organometallic compound or conjugate containing one or more atoms of iron, copper or other transition metals that can catalyze the conversion of superoxide, perhydroxyl radical or hydrogen peroxide into other compounds, yielding products that may include hydroxyl radicals and other reactive oxygen species. The hydroxyl radicals generated locally by the Fenton complex metal atoms, have their greatest effect very close to these metal atoms because of their highly reactive nature, which also enables them to chemically modify a wide range of molecules, including membrane lipids, polypeptides. RNA. DNA, carbohydrates and other biological molecules. A combination of the selenium-carrier and Fenton complex, may either be simultaneous, or sequential administration in vivo, whichever order is found to be advantageous. In addition, the dosages and molar ratios of the selenium-carrier and chosen Fenton complex may require experimental optimization. Again, the organo-metallic compound of may be an iron, copper or other transition metal covalently attached to, tightly chelated by, coordinated with or enclosed by a small organic molecular framework. These small organic molecular frameworks may include synthetic metal coordinating compounds, or naturally occurring siderophores coupled to the peptide or peptidomimetic targeting molecule. These frameworks would be loaded with the appropriate tightly bound or encaged metal ion prior to final purification, storage and in vivo administration. The organo-metallic compound might be chemically modified or extended to produce an organic molecule that can bind to either RNA. DNA or both by its ability to intercalate between the bases of the polynucleotide. The RNA or DNA intercalating organo-metallic can be designed or selected to be membrane-permeable, and able to combine with the genetic material of cells, thereby targeting the production of hydroxyl radical and its damaging effects to the genetic material of the cancerous or abnormal cell. Alternatively, the RNA or DNA intercalating organo-metallic can be designed or selected to be membrane-impermeable, such that it will preferentially bind to the genetic material of cells that will selectively import the carrier, have sustained membrane damage from superoxide produced by the targeted selenium atom, or are otherwise inherently leaky to this Fenton complex agent, thereby targeting the production of hydroxyl radical and its damaging effects to genetic material of the cancerous or abnormal cells. This type of Fenton complex would provide an additional level of control to avoid targeting free radical damage to genomic DNA or other intracellular components of healthy human or mammalian cells. As with previous examples, organo-metallic compound can be chemically linked to an antibody, protein, peptide or peptidomimetic compound designed as previously described such that it will bind with high selectivity to the same protein that is the binding target of the antibody, protein, peptide or peptidomimetic used to target selenium to the surface of the cancer cell or abnormal cell, as described in claim 1. The antibody, protein, peptide or peptidomimetic of the Fenton complex may be identical to the antibody, protein, peptide or peptidomimetic synthetic carrier that is conjugated to the selenium compound described above.

In cases where the protein or other molecule that selectively binds to this carrier exists as a dimer, trimer, or higher level multimer, a significant fraction of superoxide-generating selenium atoms will be positioned within nanometers of Fenton complex metal atoms, which will convert these into highly reactive hydroxyl radicals. The peptide or peptidomimetic of the Fenton complex may have a different binding site specificity from the peptide or peptidomimetic synthetic carrier that is conjugated to the selenium compound described above. In cases where the carrier selectively binds to a different and non-competing site on the same target protein, superoxide will be generated within nanometers of the Fenton complex metal atoms, which will convert these into highly reactive hydroxyl radicals. Finally, in cases where the Fenton complex and selenium-conjugate selectively bind to two different proteins that are localized to the same cell and plasma membrane domain, same viral envelope or same viral capsid. Fenton complex metal atoms will convert a fraction of the selenium-generated superoxide into highly reactive hydroxyl radicals.

For in vivo administration it may be advantageous to use higher dosages of a relatively lower toxicity iron-based Fenton complex in conjunction with a smaller dosage of the selenium conjugate. In cases where the target is a virus defined by the combination of two independent peptide binding sites on one large surface protein, or two separate but intermixed proteins on the surface of the envelope or protein capsid, targeted viral inactivation with two different carriers has the potential for a greater degree of selectivity than methods based on a single binding site.

Another embodiment of a Fenton complex is an iron-filled ferritin complex containing roughly 4500 iron atoms brought into the proximity of the active selenium-conjugate by a targeting peptide, peptidomimetic, protein or antibody. The rationale is that by bringing ferritin close to the targeted selenium, superoxide radicals produced by selenium would diffuse into the large ferritin-iron complex, where the iron would catalytically convert them to highly reactive hydroxyl radicals. These hydroxyl radicals would eventually damage the ferritin shell, leading to local release of iron in the vicinity of the selenium-targeted cells. The local presence of many soluble iron atoms would greatly enhance the local effectiveness of the targeted selenium. This enhanced Fenton or Fenton-like effect would be transient, since the released iron would eventually be sequestered and removed by transferrins normally present in serum and in interstitial spaces around tissues. However, it would significantly enhance cell killing by the targeted selenium atoms in regions where there are many cancer or abnormal cells. The Fenton complex would be constructed by using a chemical linker to covalently cross-link a single ferritin complex to each targeting peptide, peptidomimetic, protein or antibody. This method would use purified ferritin-iron complex of human origin, either from organs, cultured human cells, or by expression of human recombinant heavy and light chain proteins followed by self-assembly of the iron-ferritin complex. Alternatively, ferritin-iron complex could be obtained from an animal source that is, for practical purposes, immunologically compatible. Alternatively, the cell-targeting carrier of the Fenton complex peptide, protein, antibody or peptidomimetic would be extended by the addition of an antibody or designed peptide that would confer the additional ability to bind selectively to human ferritin protein. After in vivo intravenous administration, this would allow the targeting carrier to first bind to a circulating ferritin-iron complex, which is found normally in blood at reasonable abundance. The targeting carrier would then bind to the cancerous or abnormal cell, thereby bringing an iron-ferritin complex near some of the targeted selenium-carrier molecules.

Summary. The compositions and methods of the present invention have the advantage of the simplicity of a modular design to target many different viruses, bacteria, protozoal pathogens and cell types. The required agents also take advantage of the economy and speed of well-established chemical manufacturing methods. Also, the durability of the inactive selenopeptide compound and the Fenton complex compounds at ambient temperature contribute to simplified storage and rapid distribution.

### Example 2. Effect on Cancer Cells

A new hormono-conjugate was developed in order to provide a novel approach to prostate cytotoxicity. This will be a selenium adduct of dihydrotesterone (Se-DHT). The structure is shown below:

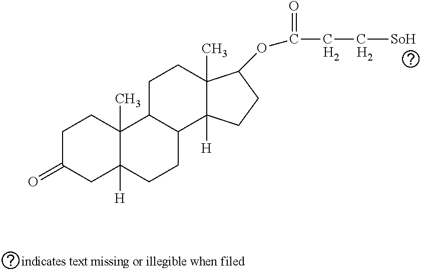

Se-DHT was used in a series of experiments to test its ability to target and kill prostate epithelial and prostate cancer cells while not affecting other types of cells.

The inventors tested the compound, SeDHT, against prostate cancer cell lines and found that it will kill both PC-3 and DU-145 cells at less than 10 micromolar concentration. This data is shown in FIG. 3. The plot measures percentage growth as established by cells with no added SeDHT vs varying concentrations of SeDHT. PC-3 cells are represented by the solid line, while the DU-145 cells are the dotted line.

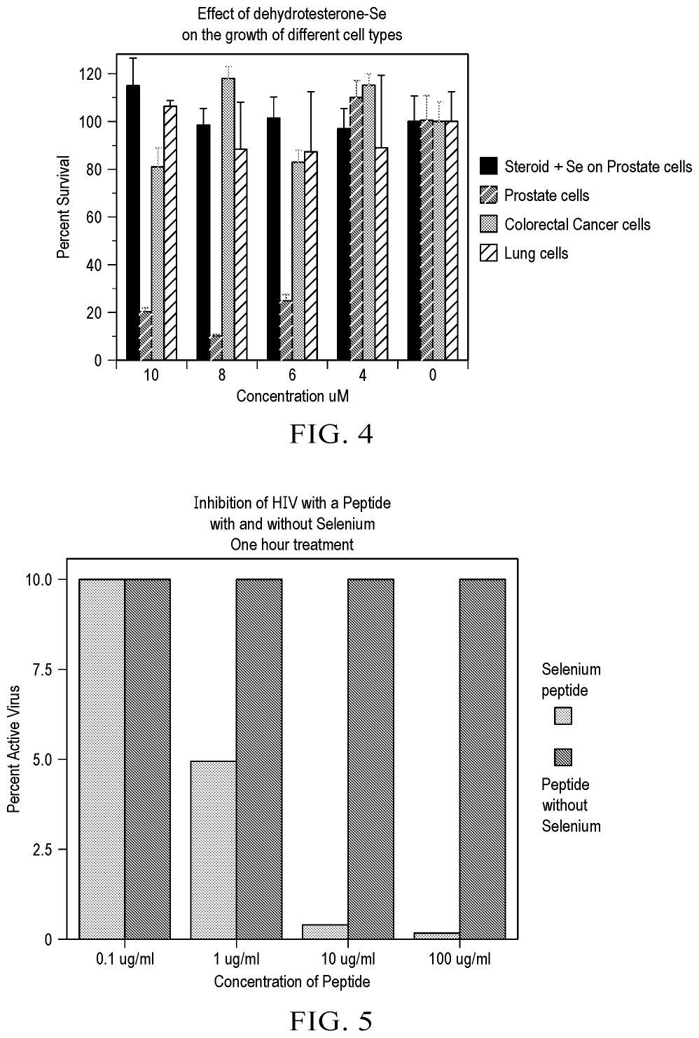

FIG. 4 shows the effect of the selenium-steroid conjugate and selenium plus steroid (not conjugated) on normal prostate cells, colorectal cancer cells and normal human lung cells. Only the prostate cells are sensitive to the selenium-steroid conjugate at up to 10 μM. The non-conjugated selenium is not toxic to any of the cells tested at 10 μM.

### Example 3. Selenopeptides Specific for HIV Virus

A 12 amino acid CD4-derived selenopeptide, which had been originally designed by another lab to bind to the viral gp120 protein, and competitively block HIV binding to its receptor, CD4 (37). They found 50% infection blocking activity at a peptide concentration of 32 μM (micromoles/liter), corresponding to about 50 μg/ml (37). Although this appeared initially promising, in vivo competitive blocking of HIV infection required much higher and clinically impractical serum concentrations of peptide. However, using the seleno-peptide of the present invention, HIV experiments show that coupling selenium to the 12-mer peptide increases its effectiveness at preventing infection by 50-fold, with 50% inactivation at 1 μg/ml, and over 95% inactivation at 10 μg/ml.

FIG. 5 shows the inactivation of HIV virus infectivity by CD412-mer selenopeptide. Virus was incubated with different concentrations of selenopeptide Se-TYICEVEDQKEE (SEQ ID NO: 3) in medium containing 0.25 mM reduced glutathione for one hour, followed by 100-fold dilution in culture medium and infection of cells. For this 1.7 kD peptide, half maximal inactivation of virus was achieved at 1 μg/ml=590 nM (nanomolar) peptide during the 1 hour pre-incubation, corresponding to 5.9 nM peptide in the medium during infection.

A solution containing 0.25 mM reduced glutathione and 10 micrograms per ml of seleno-propionyl-peptide was found to inactivate 95% of HIV virus within 1 hour. 50% inactivation was achieved using 1 microgram per ml of seleno-peptide. Without selenium, the gp120-binding 12-mer peptide had no significant effect even at 100 μg/ml, the highest concentration tested. This data shows that relatively low concentrations of selenopeptides can be highly effective at inactivating HIV and potentially other membrane-enveloped RNA viruses.

SARS-COV-2. A seleno-peptide specific for SARS-COV-2 can be designed as was done for HIV. Phage display can be used to isolate peptides that are specific for SARS-COV-2. To increase their activity, the peptides are modified to form the selenium-peptide conjugate. The activity of the selenium-peptide conjugate is then tested in vitro and in vivo for anti-viral activity.

### Example 3. Organo-Selenium Peptide to Develop New Antimicrobials that Target a Specific Bacteria

Selenopeptides specific for bacteria and HIV virus. Bacteriophage display was used to develop a 12-mer peptide that bound specifically to the F1 antigen, a protein present on the surface of the bubonic plague bacterium Yersinia pestis. F1 antigen was expressed in E, coli. A selenopeptide derivative of this 12-mer was prepared, and a 10 μM concentration of this selenopeptide shown, to selectively kill the F1-expressing bacteria, decreasing the titer by several logs in 2 hours.

Peptide 8—Ser Ser Leu Thr Leu Ala Pro Phe Ser Trp Ser Leu SEQ ID NO:2.

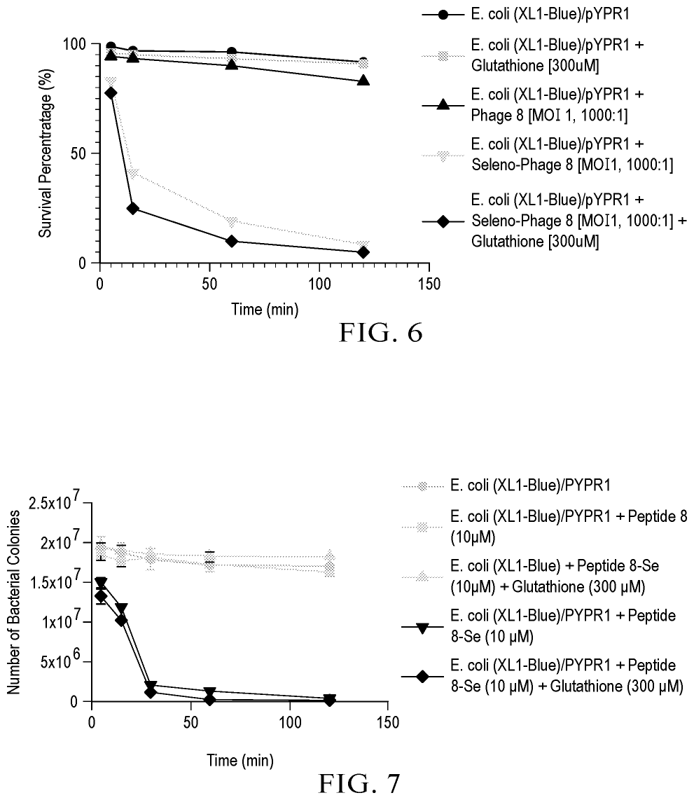

FIG. 6 is a graph that shows the effect of selenium-attached phage #8 on the viability of E. coli XL1-blue/pYPR1 under added oxygen. Survival of the Y, pestis F1-antigen-expressing E, coli XL1-blue/pYPR1 strain in the presence of selenium-labeled phage #8, an MOI (multiplicity of infection) of 1000:1, and 21% oxygen, with and without reduced glutathione (300 microM) in PBS. The control contained bacteria pYPR1 in PBS. Other controls included pYPR1 with either reduced glutathione or phage #8 in PBS. The experiments were carried out at room temperature for two hours. After several time points, aliquots of the solutions were serially diluted and plated on LB agar supplemented with 100 micrograms/mL carbenicillin and c.f.u, was counted after overnight incubation. Multiplicity of infection (MOI) is the ratio of phage to bacteria.

FIG. 7 is a graph that shows the effect of seleno-peptide #8 (10 microM) on the E, coli XL1-blue/pYPR1strain, with or without glutathione, under added oxygen. One control contained only bacteria XL1-blue/pYPR1. Other controls included XL1-blue/pYPR1 with either reduced glutathione (300) microM) or peptide #8 (10) microM) without selenium. Values represent the mean from three independent experiments. E, coli (XL1-blue) is the parent strain without the plasmid for the F1 antigen.

It is important to note that the E, coli (XL1-blue) parent strain, without the plasmid for the F1 antigen, shows no killing by peptide-8 with selenium. This indicates that the selenium peptide is only able to kill bacteria that express the F1 antigen on their surface.

4.1. Bacterial Strains, Media, and Reagents. Escherichia coli (E, coli) XL1-blue/pYPR1 expressing Yersinia pestis (Y, pestis) F1 from the cloned caf operon and purified Y, pestis F1 protein were described earlier [18]. The cloned plasmid was obtained from T. Schwan, Rocky Mountain Laboratories, Hamilton, Montana. The E, coli ER2738 strain and PhD-12 phage-display kit were purchased from New England Biolabs, Inc. (www.neb.com, accessed on 20 May 2021). The Escherichia coli XL1-blue parent strain was obtained from Stratagene (Stratagene, La Jolia, CA, USA). The E, coli were maintained at 4 degrees C., in Luria Broth (LB) medium containing 30% (v/v) glycerol.

Antibiotics were used at the following concentrations in the LB medium: 20 microg/mL tetracycline for E, coli ER2738 and 100 microg/mL carbenicillin for the expression of the Yersinia pestis F1 antigen in E, coli XL1-blue/pYPR1. In addition, the E, coli ER2738 strain was grown with shaking at 37 deg C., in LB medium or on LB agar supplemented with 20 microg/mL tetracycline. The E, coli XL1-blue parent strain was grown with shaking at 37 deg C., in LB medium or on LB agar. In contrast, the E, coli XL1-blue/pYPR1 strain was routinely grown with shaking at 37 deg C., in LB medium or on LB agar supplemented with 100 microg/mL carbenicillin to maintain the pYPR1 plasmid. E, coli ER2738 was used to grow the phage.

The LB medium contained 10 g/L Bacto-Tryptone (#211705, BD BioSciences, San Jose, CA), 5 g/L yeast extract (#212750, BD BioSciences, San Jose, CA, USA), and 5 g/L NaCl (#S271-3, Fisher Sci., Hampton, NH, USA). In contrast, LB/IPTG/X Gal Plates contained the basal LB medium with 15 g/L agar (#214010, BD BioSciences, San Jose, CA, SUA), 50 microg/L IPTG/XGal (#15529-019, Invitrogen, Carlsbad, CA, USA), and 40 microg/L XGa (#15520-018, Invitrogen). The LB agar was composed of the LB basal medium and 15 g/L agar. The blocking buffer contained 0.1 M NaHCO₃(pH 8.6), 5 mg/mL BSA, and 0.02% NaN3, which was filter-sterilized and stored at 4 deg C. The PEG/NaCl solution contained 20% (w/v) polyethylene glycol-8000 and 2.5 M NaCl, which was autoclaved and stored at room temperature. The iodide buffer [10 mM Tris-HCl (pH 8.0), 1 mM EDTA, and 4 M Nal. The solution was stored at room temperature in the dark], TBS solution, [50 mM Tris-HCl (pH 7.5) and 150 mM NaCl. Autoclave, store at room temperature], and Agarose Top [per liter: 10 g Bacto-Tryptone, 5 g yeast extract, 5 g NaCl, 1 g MgCl2 6H2O, and 7 g agarose. The solution was autoclaved and dispensed into 50 mL aliquots, stored solid at room temperature, and melted in a microwave as needed] were used in the experiments.

Preparation of Cells for Infection. A single colony of E, coli ER2738 from a stock plate was grown in twenty milliliters of LB medium at 37 deg C., with shaking at 225 rpm overnight. Aliquots of these cells were used immediately for transfection after each round of biopanning.

Biopanning and Screening of Phage-Displayed Peptides against Purified Y, pestis F1 Antigen To select specific peptide sequences with high Y, pestis specificity and affinity, the inventors screened a random peptide filamentous (M13) phage display library (PhD-12) for their ability to bind to the pure F1 antigen. Biopanning was performed following the method described in the Instruction Manual (pH D.-12 phage display, New England Biolabs Inc.) using TBS [50) mM Tris-HCl (pH 7.5) and 150 mM NaCl]. Next, 1.5 mL of 100 microg/mL of purified Y, pestis F1 antigen (obtained from the CDC in Denver, Colorado) in 0.1 M NaHCO₃(pH 8.6) was incubated at 4 deg C., overnight in a 35×10 mm polystyrene dish (code 25060-60; Corning, New York, NY, USA). The Y, pestis F1-antigen-coated dish was blocked for 2 h at 4 deg C., with blocking buffer [0.1 M NaHCO₃ (pH 8.6), 5 mg/mL bovine serum albumin (BSA), and 0.02% NaN3], and then washed six times with TBS containing 0.5% Tween 20 (TBS/T). After washing six times with TBS/T, 10/microliters (2×10¹¹ phage) of the pH.D.-12 library (#E8100S) was dissolved in 1 mL 0.5% TBS/T, and the mixture was added to the dish coated with F1. The F1-coated dish with the phage mixture was then incubated for 2 h at 4 deg C. After incubation, it was washed six times with 0.5% TBS/T. The bound phage was eluted with 1 mL elution buffer (0.2 M glycine-HCl (pH 2.2) and 1 mg/mL BSA) for 10 min. The eluate was neutralized with 150 microliters 1 M Tris-HCl (pH 9.1). The phage titers of serial dilutions of phage eluates were determined by plating on lawns of E, coli ER2738, as described below in the phage tittering section.

Final Phage Preparation and DNA Extraction. After tittering the eluate of the 3rd round of biopanning, isolated phage clones were amplified. Individual phage clones (blue colonies) were grown from the clones that bound to the Y, pestis F1 antigen and single-stranded DNA through the following procedure. An overnight culture of the host (ER2738) strain was diluted at 1:200 in LB/Tet. The phage plaques were tooth-picked and transferred to 1 mL aliquots of a 1:200 dilution of a fresh overnight culture of E, coli ER2738 and placed in 13×100 mm glass tubes. The E, coli ER2738 were then grown with shaking for 8 h at 37 deg C., in a water bath, after which the culture was transferred to 1.5 mL microfuge tubes. This was then microcentrifuged for 5 min at 15.000 rpm. The upper 80% of the supernatant was transferred to a fresh 1.5 mL microfuge tube, where half (400 microliters) of the supernatant was kept as the final phage preparation while the other half was used to extract DNA for sequencing.

Next, 200 microliters of 20% PEG with 2.0 M NaCl was added and allowed to sit for 10 min at room temperature. This solution was microcentrifuged for 10 min at 15.000 rpm and the supernatant removed. The pellet was resuspended in 100 microliters iodide buffer (10 mMTris-HCl, ImMEDTA, 4MNaI, and pH 8.0) and precipitated with 250 microliters of 95% ethanol. The mixture was incubated at room temperature for 10 min, after which it was remicrocentrifuged for 10 min at 15.000 rpm, with the supernatant discarded. The pellet was washed with 500 microliters of 70% ethanol and dried briefly under vacuum. The pellet was then resuspended in 30 microliters of sterile water. The phage DNA sequences were determined at the Center for Biotechnology and Genomics Core Facility (TTU, Lubbock, TX, USA) using the −96 gIII sequencing primer (New England Biolabs #1259). The N-terminal amino acid sequences of the gene III products of the selected display phage were deduced from their DNA sequences.

Phage Binding Analysis by the Spun Cell ELISA. In order to identify the phages that recognize the F1 antigen on the surface of a bacterial cell, we used the Spun-Cell ELISA assay. This assay was employed to target the portion of the F1 antigen that is displayed on the surface of an E, coli cell. The procedure was carried out as previously described by Benhar et al. [19]. Briefly, the E, coli XL1-blue parent strain and the Y, pestis F1-antigen-expressing E, coli XL1-blue/pYPR1 strain were grown overnight at 370C in LB medium supplemented with an appropriate antibiotic. Bacterial cultures were diluted to an OD 600 nm˜0.2 (1×108 cells/mL). The cell culture suspension was then cleared by centrifugation. The cell pellet was then washed twice with PBS and resuspended in 0.2% TBS/T (Tris-buffered saline (TBS; pH 7.4) and 0.2% Tween 20). A 1 mL aliquot of the bacterial dilution was transferred to each 1.5 mL microfuge tube for analysis of the surface display of the antigen. Phage clones from the previous selection process (1×1011) were added to each 1.5 mL microfuge tube, and the mixture incubated at room temperature for 1 h. Cells were then centrifuged and washed. For detection of phage binding, we used an HRP-conjugated anti-M13 antibody (Amersham Pharmacia Biotech. #27-9420-01) that was diluted to 1:5000 in blocking buffer. The cell mixtures were incubated for one hour on ice and washed five times with 0.2% TBS/T by repeated centrifugation and resuspension. Afterward, 100 microliters of HRP 3,30,5,50-tetramethylbenzidine (TMB) substrate (Sigma, St. Louis, MO, #T0440) was added to each 1.5 mL microfuge tube and the reaction allowed to stand for 10 min at room temperature for detection. The color development was terminated with 100 microliters Stop Reagent for TMB Substrate (Sigma, #S5814). The absorbance was recorded at 450 nm.

Methodology for Labeling Phage with Selenium. A phage clone, phage #8, chosen as the best binder from the Spun-Cell ELISA assay, was amplified in 200 mL LB-Tet (20) micrograms/mL) medium for 8 h by the addition of 2 mL of the E, coli ER2738 strain from an overnight culture. The resulting phage was then purified from the supernatant (Ph.D. Phage Display Libraries Instruction Manual-NEB #E8100S, E8101S, E8110S, E8111L, E8120S). The amplified eluate was then reamplified again until the volume of 1-5 mL (˜1 1013 phage/mL) was reached. This solution would then be used in the covalent attachment of a selenium compound. The selenium compound (cyanatoseleno-acetic acid; NCSeCH2COOH: 16.4 mg) was dissolved in 1 mL of MES (2-(N-morpholino)ethanesulfonic acid) buffer in a 10-mm-diameter tube. Next. 22 mg of N-hydroxysulfosuccinimide (Sulfo-NHS) and 20 mg 1-ethyl-3-(3-dimethyl aminopropyl) carbodiimide (EDC, Pierce Chemical, Rockford, IL, USA) were added to the solution.

The reaction was then carried out with stirring at room temperature for 1 h. Next, 10 microliters of the reaction mixture was added to every 1 mL phage (˜1×10¹³ phage/mL) and stirred at room temperature for 30 min. The mixture was then transferred to a membrane tubing (MWCO (6-8000), Spectrum) and dialyzed in 5 L of water, which was changed every 2 hours at least 5 times. This process was done to eliminate unbound selenium compounds and other excess chemicals. Then, 1 mL of seleno-phage was purified from the supernatant by precipitation with 1/6 volume of PEG/NaCl (20% polyethylene glycol-8000 and 2.5 M NaCl). The precipitate was collected by centrifuging at 10,000 rpm at 4 deg C. (15 min). Finally, the pellet was dissolved in 1 mL of PBS. This seleno-phage stock was used in the killing assay.

To determine the presence of selenium on phage #8, 5 mL of dialyzed seleno-phage (˜1×10¹³ phage/mL) was precipitated with 2.7 mL of PEG/NaCl (20% polyethylene glycol-8000 and 2.5 M NaCl). The pellet was then dissolved in 200 microliters PBS. Next, 50 microliters was used in the chemiluminescent assay to confirm the presence of selenium. To determine the presence of selenium on peptide #8, 1 mg of selenopeptide #8 was dissolved in 1 mL of water. Finally, 100 microliters of the selenopeptide #8 solution was used in a quantitative fluorescent assay described Painter, E. P. The Chemistry and Toxicity of Selenium Compounds, with Special Reference to the Selenium Problem. Chem. Rev. 1941, 28, 179-213.

Bacterial Killing Assays with the Selenium-Labeled Phage and Peptides. The antibacterial activity of the phage and peptides covalently labeled with selenium was determined. The overnight E, coli XL1-blue and E, coli XL1-blue/pYPR1 bacterial cultures were diluted to the optical density of 0.2 (˜108 bacteria per mL) at 600 nm (A600 nm), and cells were collected by centrifugation and washed twice with PBS. The cells were resuspended in the same volume of PBS, and aliquots equivalent to 1 mL were transferred to 1.5 mL microfuge tubes. The seleno-phage or seleno-peptides were added to a bacterial suspension, with a multiplicity of infection (MOI) of 1000:1 (phage per bacterium) or 10 micromolar, respectively. In designated tubes, glutathione was added to a final concentration of 150 or 300 micromolar, and the mixtures were incubated at room temperature. In the other experiments, extra oxygen was supplied from air by an air pump (Pipet Aid. Drummond Scientific Co. #262). Briefly, a flexible airline tube was connected to an air pump. The flexible airline tube was then connected to multiway gang valves. Each outlet from these multiway gang valves was plugged with a needle (B-D. #305185). The speed of flowing air was adjusted through the valves of the above multiway gang valves so that gentle bubbles were observed. At different time intervals, samples were collected, and serial dilutions (1:100) were performed.

Viable cell counts were determined by plating serial dilutions on LB plates, with or without appropriate antibiotics, after 24 h of incubation at 37 deg C.

Peptide Synthesis. The peptides, which represented the sequences obtained from the phage libraries, were synthesized on a small scale. Briefly, the synthesis of peptides was started from carboxy-terminal amino acid. First. 1 g resin (sometimes with the first amino acid on it) was transferred by DMF (dimethylformamide) to the peptide synthesizer and allowed to stand in the DMF for 30 min. The DMF was removed completely under vacuum. Second. 10 mL of 20% piperidine in DMF was added, and the mixture bubbled with N₂ for 5 min. The solution was then drained, after which 10 mL of more piperidine solution was added, and the mixture bubbled for 20 min. The mixture was drained thoroughly and washed 3 times with DMF and 2 times with isopropanol. A small amount of the solution was used in a Ninhydrin test (1.2.3-indantrione monohydrate or triketohydrindene hydrate) to detect free amino groups in the presence of blue color. The rest of the beads were washed three more times with DMF. Two equivalents of amino acid (in 7 mL DMF) were added and bubbled with nitrogen gas. Two equivalents of PyBop (in 3 mL DMF) and four equivalents of pure DIEA (N N-diisopropylethylamine) were then added and bubbled with nitrogen gas for 1 h. The mixture was then drained and washed three times with DMF. A small amount of the beads was used in the Ninhydrin test, which produced a yellow color. The Fmoc (9-Fluorenylmethoxycarbonyl) group was removed from the amine terminal of a growing peptide chain in basic conditions (usually 20% piperidine in DMF). The procedure was repeated until the last desired amino acid was added. The mixture was then washed three times with DMF, three times with CH₂Cl₂, and three times with methanol. The mixture was then drained thoroughly. The resin was transferred to a vial, freeze-dried overnight, and then stored in the freezer. The peptides were then prepared and dissolved at 1 mg/mL concentration in water (pH=8.0).

Peptide #8 Structure and Amino Acid Sequence Analysis. The synthetic peptide #8 was purified by high performance liquid chromatography (HPLC). The purification was achieved by reverse-phase column with linear gradients of 0.1% TFA in water and 0.075% TFA in CH3CN at a 1.5 mL/min flow rate: the gradient range (20% CH₃CN to 100% CH₃CN) vs. gradient time (40) min) vs. gradient steepness (2%/min). The molecular weight was confirmed by mass spectroscopy (MW=1307.692, Colorado State University, Fort Collins CO). The amino acid sequence of peptide #8 was determined and confirmed by a protein sequencing system at the Center for Biotechnology and Genomics Core Facility (TTU, Lubbock, TX, USA). The peptide's amino acid sequence was also confirmed by two-dimensional NMR spectroscopy (2D-NMR).

Seleno-Peptide Synthesis. For this synthesis, a BrCH₂CH₂CO-group is coupled to the terminal amino residue of the peptide on the resin obtained above. Twelve equivalents of BrCH₂CH₂COOH in dry CH₂Cl₂ were added and allowed to dissolve. The reaction flask was closed, and the solution was cooled with ice water. A solution of DCC (six equivalents) in dry CH₂Cl₂ was added and stirred for 20 min. The white solid was filtered off, and then the solvent was removed by rotary evaporation to obtain the anhydride. First, resin and DMF were mixed and allowed to stand for 30 min, after which the resin was drained thoroughly. The anhydride was dissolved in DMF, and the solution was added to the resin. The mixture was bubbled with nitrogen gas for an hour. The mixture was then washed three times with DMF, three times with CH₂Cl₂, and three times with methanol. The solvents were drained thoroughly. The resin was then transferred to a vial, processed by freeze-drying overnight, and stored in the freezer. The peptide was then cleaved from the resin. The dry resin was placed in a vial, and TFA (trifluoroacetic acid) added at the ratio of TFA/TIS (triisopropyl-silane)/water of (95:2.5:2.5), using 15 mL/g resin. The mixture was allowed to stand at room temperature with occasional swirling for 2 h. The resin was then removed by filtration under reduced pressure and washed twice with TFA. The TFA was evaporated by a rotary evaporator, and an 8- to 10-fold volume of cold ether was added (dropwise) to the filtrates. The suspension was then transferred to a clean 1.5 mL microfuge tube, sealed, and centrifuged. Ether was decanted from the tube. An additional 8- to 10-fold volume of cold ether was added to wash scavengers, followed by centrifugation. The residual solid was dissolved in a CH₃CN/H₂O (50/50) mixture and then lyophilized. Next. 20 mg of crude bromo-peptide and 20 mg potassium selenocyanate were dissolved in 1 mL of DMF. The reaction was allowed to proceed at room temperature for 24 h. Then. 1 mL of water and 20 mg of ammonium chloride were added to quench the excess seleno-cyanate. The seleno-cyanate peptide was then purified by reverse-phase HPLC. The lyophilized peptide was stored by freezing at −20 deg C. All seleno-peptides synthesized were tested for superoxide radical generation capability by a chemiluminescent assay (see below). In addition, seleno-peptides were prepared at 1 mg/mL concentration in water (pH=8.0). The conformation was studied and compared to that of peptides without selenium attachment by circular dichroism.

Chemiluminescent (CL) Assay. A chemiluminescent assay was used to determine the activity of selenium on the phage and peptides. The control chemiluminescent (CL) assay cocktail, without substrates or GSH (reduced glutathione), was made using 0.05 M sodium phosphate buffer (pH=7.4) and 20 microliters lucigenin/mL from a stock solution of 1.0 mg/mL lucigenin in distilled water. The assay cocktail with thiol contained GSH (1.0 mg/mL): 50 microliters of seleno-phage or phage (107 phage/mL) or 50 microliters of seleno-peptide or peptide (10 micrograms/mL) were added to 500 microliters test aliquots of the control or thiol-containing assay cocktail. Chemiluminescent (CL) data were recorded in integrated units over a period of 5 min.

Competitive Inhibition ELISA. To check the binding of the peptide to target proteins, a competitive inhibition ELISA assay was employed. The E, coli XL1-blue/pYPR1 and XL1-blue strains were grown overnight at 37 deg C. under the conditions described above. Bacterial cultures were diluted to OD600 nm˜0.2 (˜108 cfu/mL), and 1 mL of the bacterial dilution was transferred to each microcentrifuge tube. The pYPR1 and XL1-blue cells were used to determine the binding competition between peptide #8 and the F1 antibody for the F1 antigen on pYPR1 cells. The pYPR1 cells were then first incubated with either peptide #8 (1 micromolar) or the F1 antibody (0.25 micrograms/mL) for an hour at 4 deg C. The cells were centrifuged and washed. The F1 antibody (0.25 micrograms/mL) and peptide #8 (1 micromolar) were then added, whereby the suspensions were incubated at 4 deg C., for an hour. Cells were centrifuged and washed 5 times. The F1 antibody was detected using an anti-mouse monoclonal IgG antibody that was HRPconjugated (1:3000). The cell mixtures were incubated for one hour on ice and washed five times with 0.2% TBS/T by repeated centrifugation and resuspension. Next. 100 microliters of 3.30.5.50-tetramethylbenzidine (TMB) substrate (Sigma. #T0440) was added to each 1.5 mL microfuge tube. The reaction was allowed to stand for 10 min at room temperature for detection. The color development was terminated with 100 microliters Stop Reagent for TMB Substrate (Sigma, #S5814), and the absorbance was recorded at 450 nm.

Extraction of F1 Antigen from E, coli. The extraction of the F1 antigen was performed as previously described by Simpson, W. J.: Thomas, R. E.: Schwan, T. G. Recombinant Capsular Antigen (Fraction 1) from Yersinia Pestis Induces a Protective Antibody Response in BALB/c Mice. Am. J. Trop. Med. Hyg. 1990, 43, 389-396. The E, coli cells carrying pYPR1 were grown overnight in 20 mL LB medium supplemented with carbenicillin (100 micrograms/mL) and then harvested by centrifugation using 10,000 rpm for 5 min in a Beckman rotor. Next, 80% of the supernatant was transferred to a fresh tube, while the remaining supernatant was transferred to another tube. Above the cell pellet was a less dense, flocculent material, which was recovered with the supernatant. This material was solubilized in SDS-PAGE buffer, boiled for 5 min, and loaded into a 14% preparative SDS-polyacrylamide gel. The cell pellet was washed twice gently in 2 mL PBS, repelleted by centrifugation, and resuspended in 2 mL PBS. Following electrophoresis, the gel was stained with water-based Coomassie Blue R270 to identify the 14 to 17 kDa band, which corresponded to the F1 antigen. The presence of the F1 antigen in the supernatant was subsequently confirmed by immunoblot.

SDS-Page and Western Blot Procedure. The molecular weight and relative amount of F1 antigen produced by recombinant E, coli pYPR1 were determined by SDS-polyacrylamide gel electrophoresis (SDS-PAGE) and Western blot. The pYPR1 was grown from a single colony in 20 mL LB medium supplemented with 100 micrograms/mL carbenicillin for 24 h and then harvested by centrifugation, using 10,000 rpm for 5 min in a Beckman rotor. Approximately 80% of the supernatant was transferred to a 1.5 mL fresh tube. The remaining supernatant was transferred to another tube. Then, 1 mL of the supernatant factions was treated with 100 microliters trichloroacetic acid (TCA) and incubated on ice for an hour. The TCA-treated factions were then centrifuged for 5 min and the supernatants discarded. The pellets were washed with TBS [50 mM Tris-HCl (pH 7.5) and 150 mM NaCl]. The pellets were resuspended in 100 microliters sterile water and 100 microliters 4× sample buffer.

For supernatant samples without TCA treatment, 100 microliters supernatant was transferred to a 1.5 mL fresh tube, to which 100 microliters 4× sample buffer was added. The purified F1 antigen sample was prepared by mixing 50 microliters from purified F1 stock solution (1 mg/mL) with 50 microliters sterile water and 100 microliters 4× sample buffer. Cell pellet samples were prepared by suspending the pellets in 2 mL PBS. Next, 100 microliters was then transferred to a 1.5 mL microfuge tube, to which 100 microliters 4× sample buffer was added. The XL1-blue sample was prepared using 1 mL of overnight culture, which was transferred to a 1.5 mL microfuge tube. The cells were centrifuged, and the supernatant was discarded. The cell pellet was resuspended in 100 microliters sterile water and 100 microliters 4× sample buffer. All the samples were boiled for 5 min, and 20 microliters of the denatured sample was loaded and run on a 14% SDSPAGE.

The resulting gel proteins were transferred to a PVDF membrane (BIO-RAD, #14098) using the Trans-Blot SD Semi-Dry Transfer Cell (Bio-Rad, #221BR 17035). The membrane was blotted with blocking solution (2% BSA and 7.5% nonfat dry milk, 0.2% Tween-20 in TBS (60 mg/mL NaCl, 1.21 mg/mL Tris-base, and pH=7.4)) for an hour with gentle rocking at room temperature.

The membrane was then washed 5× in wash buffer (0.2% Tween-20 in TBS), 10 min with gentle rocking per wash. Afterward, the primary antibody (diluted 1:4000 mouse monoclonal IgG F1 antibody (1 mg/mL) in wash buffer with 0.25% BSA and 2% nonfat dry milk) was added and rocked gently for 1 h. The membrane was then washed 5× in wash buffer (0.2% Tween-20 in TBS), 10 min with gentle rocking per wash. Then, the secondary antibody (diluted 1:3000 anti-mouse monoclonal IgG antibody (1 mg/mL) in wash buffer with 0.25% BSA and 2% nonfat dry milk) was added and rocked gently for 1 h. The membrane was then washed 5 more times in wash buffer, then covered in Pierce Super Signal West Pico Chemiluminescent Substrate (5 mL peroxide solution and 5 mL luminol/enhancer solution) (#34080) for one minute. The membrane was then exposed to Blue Sensitive Autoradiographic Film (Marsh Bio Products #75590) for 3 min and developed.

Statistical Analysis. Statistics were calculated using InStat (Graph Pad Software, San Diego, CA). The p-values were calculated according to the paired t-test, where a significant value was p<0.5.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A method of making a selenium-carrier conjugate for in vivo administration comprising:
covalently attaching a selenium compound that reacts in vivo with naturally occurring reduced thiols and oxygen to produce superoxide and perhydroxyl radicals to a synthetic targeting carrier to form the selenium-carrier conjugate, wherein the selenium-carrier conjugate is stabilized against enzymatic degradation and clearance from the body and the synthetic targeting carrier binds target.

2. The method of claim 1, wherein the synthetic targeting carrier binds with high specificity to an external domain of a targeted protein or membrane protein on the surface of an animal virus, cellular microbe, or mammalian cell.

3. The method of claim 1, wherein at least one of:
the selenium-carrier conjugate produces superoxide radicals and their protonated derivative, the perhydroxyl radical, that locally damages lipids in viral envelopes or proteins in viral capsids to inactivate animal viruses or kill one or more bacteria, fungi, protozoans, cancerous or otherwise disease-causing mammalian cells and selectively kills infected cells infected with the one or more membrane-enveloped viruses;
wherein the synthetic targeting carrier for selenium is a chemically modified peptide or peptidomimetic molecule that specifically binds to the targeted protein, membrane protein, carbohydrate or other biological macromolecular assembly; or
wherein the selenium-carrier conjugate is a catalytically active selenium-carrier conjugate, wherein binding and dissociation rate constants of the catalytically active selenium-carrier conjugate attaches transiently to the target site, wherein the seleno-conjugate detaches and reattaches multiple times, allowing the catalytically active selenium-carrier conjugate to destroy multiple viral or cellular targets, either in vitro or in vivo.

4. (canceled)

5. The method of claim 4, wherein a peptide precursor of the synthetic peptidomimetic targeting carrier is designed by expressing and displaying a phage expression library of peptides having different amino acid sequence permutations, then selecting phage expressing peptides with the desired properties, expressing permuted amino acid peptide sequence within the polypeptide chain or the surface protein of a bacteriophage protein, and selecting the bacteriophage that bind to the targeted protein on the surface of one or more viruses or cells, or selecting completely random permutations of an amino acid peptide sequence or a partially random amino acid peptide sequence permutation encoded in the genome of and displayed as part of a protein of the corresponding capsid surface of a bacteriophage, or selecting a binding assay using a bacteriophage library.

6. The method of claim 5, further comprising at least one of:
the step of selecting peptide precursor sequences that bind at positions within a few nanometers of a membrane of the one or more membrane-enveloped viruses or cells infected with the one or more membrane-enveloped viruses to enhance the efficiency of virus inactivation or infected cell killing by their selenium-carrier conjugates, and optionally the peptide has SEQ ID NO: 1 or 2;
the step of modifying the peptide precursor sequence originally selected by bacteriophage display or another molecular display method, wherein the step of modifying comprises at least one of: extending the n-terminus and c-terminus by adding chemical groups that hinder the action of terminal peptidases; coupling carbohydrate polymers that increase solubility and extend the lifetime of the selenium-carrier complex; changing the amino acid sequence, chemically modifying the amino acids, substituting one or more peptide linkages, or substituting the peptide chain with one or more D-amino acids, wherein the modifications enhance binding affinity of the modified peptide precursor to the target and increase its resistance to proteolysis in vivo, to increase clinical effectiveness of the selenium-carrier conjugate;
the step of administering the inactive diselenide dimer intravenously, orally, topically, nasally, or through pulmonary administration by inhalation of an aqueous mist or dry powder to epithelia of the upper and lower respiratory tract, wherein the inactive diselenide dimer is converted to superoxide-generating R—Se—H monomers by in situ glutathione and other reducing compounds; or
the step of selecting selenium-carrier conjugates that selectively kill virally-infected cells by binding to excess virally-encoded envelope proteins on the plasma membrane surface during viral replication, wherein the virus is selected from genera and species Adenovirus, Poliovirus, Enterovirus, Rhinovirus, Hepatitis A virus, Yellow fever virus, West Nile virus, Dengue virus, Zika virus, Hepatitis C virus, Rotavirus, Papillomavirus, wherein if a virus is a non-enveloped viruses, production of the effective perhydroxyl radical is entirely dependent on the presence of protein and nucleic acid polyanions.

7. (canceled)

8. (canceled)

9. (canceled)

10. (canceled)

11. The method of claim 1, wherein the selenium is specifically targeted to exposed surfaces of cells for the purpose of killing these cells, wherein the cells are selected from bacteria, fungi, or protozoans, abnormal human, infected human, or mammalian cells such as cancers, or dysfunctional cells; or
the selenium-carrier conjugate is stored as a relatively stable and inactive diselenide dimer of the form R—Se—Se—R or R—Se—Se—R′, at a suitable ambient temperature that could range from −20° C., to 40° C.

12. (canceled)

13. (canceled)

14. A method of treating a human or animal patient after viral exposure or during an active viral infection comprising:
covalently attaching a synthetic targeting carrier specific for a target with a selenium compound to form a selenium-carrier conjugate;
providing the selenium-carrier conjugate by intravenous, nasal, oral, topical or pulmonary administration to the human or animal patient; and
wherein the selenium-carrier conjugate reacts in vivo with naturally occurring thiols and oxygen at the target to catalytically generate short-lived superoxide and perhydroxyl radicals.

15. The method of claim 14, wherein at least one of:
the selenium-carrier conjugate binds with high specificity to an external domain of a targeted membrane protein on a surface of a membrane-enveloped animal virus or a surface plasma membrane of a virus-infected cell;
the selenium-carrier conjugate generates superoxide radicals that transform into perhydroxyl radicals at an acidic membrane interface or a polyanionic environment with protein-encapsidated viruses; or
the perhydroxyl radicals cause oxidative damage to membrane lipids of a virus to render its protective membrane permeable, or disrupted, and to further inactivate the virus by damage to at least one of: viral proteins, viral RNA or DNA, or a viral genome.

16. (canceled)

17. (canceled)

18. The method of claim 14, further comprising providing the human or animal patient with a sufficient amount of the selenium-carrier conjugate, as formulated for in vivo administration, to effectively reduce the levels of active virus and viral infection.

19. The method of claim 14, wherein at least one of:
the synthetic targeting carrier is a peptidomimetic molecule that specifically binds to a targeted viral envelope membrane protein; or
a peptide precursor of the peptidomimetic synthetic targeting carrier is made by: expressing and displaying a peptide having an amino acid sequence permutation on a surface of a bacteriophage library, and selecting the bacteriophage that bind to a targeted transmembrane protein on a surface of one or more membrane-enveloped viruses.

20. (canceled)

21. The method of claim 19, further comprising the step of modifying a peptide to form a modified peptide precursor by at least one of: changing the amino acid sequence, chemical modifying the amino acids, substituting one or more peptide linkages, or substituting with one or more D-amino acids, wherein the modifications at least one of: enhance binding affinity of the modified peptide precursor to the target, increase resistance to proteolysis, or increase an effectiveness of the selenium-carrier conjugate in vivo, and optionally the peptide has SEQ ID NO: 1 or 2.

22. The method of claim 14, wherein the target is one or more membrane-enveloped viruses selected from a coronavirus, influenza virus, human immuno-deficiency virus (HIV), respiratory syncytial virus (RSV), or other membrane-enveloped virus; or
the target is at least one of: encapsidated non-envelope animal viruses selected from genera and species: Adenovirus, Poliovirus, Enterovirus, Rhinovirus, Hepatitis A virus, Yellow fever virus, West Nile virus, Dengue virus, Zika virus, Hepatitis C virus, Rotavirus, Papillomavirus, wherein if a virus is a non-enveloped viruses, production of the effective perhydroxyl radical is entirely dependent on the presence of protein and nucleic acid polyanions.

23. (canceled)

24. A method of treating a human or animal patient infected with a cellular microbial pathogen selected from a bacteria, fungi or protozoa comprising:
forming a selenium-carrier conjugate by covalently attaching to a selenium compound with a synthetic targeting carrier specific for the bacteria, fungi or protozoa;
providing the selenium-carrier conjugate by intravenous, nasal, oral, topical or pulmonary administration of said carrier with a sufficient amount of the selenium-carrier conjugate, as formulated for in vivo administration, to effectively reduce the levels of actively proliferating pathogen; and
reacting the selenium-carrier conjugate in vivo with naturally occurring thiols and oxygen to catalytically generate short-lived superoxide and perhydroxyl radicals;
wherein the selenium-carrier conjugate binds with high specificity to an external domain of a targeted cell membrane protein on the surface of an actively growing and proliferating cell, or a relatively inactive cyst-like form of a cellular microbe;
wherein the attached selenium-carrier conjugate generates superoxide radicals that transform into perhydroxyl radicals at the acidic plasma membrane interface, and
wherein the perhydroxyl radicals cause at least one of: sufficient oxidative damage to plasma membrane lipids to render the cell permeable and cause lysis, or sufficient damage to bacteria, fungi or protozoa DNA through mutations or chromosomal breaks to prevent significant replication of the bacteria, fungi or protozoa.

25. The method of claim 24, wherein the synthetic targeting carrier is a peptido-mimetic molecule that specifically binds to at least one of: a targeted pathogen-encoded cellular plasma membrane protein or a specific protein located on a surface of a spore or cyst form of the pathogen, or a cell wall constructed of carbohydrate and carbohydrate-peptide polymers that are specific binding targets for the selenium-carrier conjugate; or
a peptide precursor of the peptidomimetic synthetic targeting carrier is made by: expressing and displaying a library of peptides having different amino acid sequence permutations on a surface of a bacteriophage, and selecting the bacteriophages that bind to the targeted transmembrane protein on a surface of the one or more membrane-enveloped viruses.

26. (canceled)

27. The method of claim 25, further comprising the step of modifying a selected peptide precursor by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo, and optionally the peptide has SEQ ID NO: 1 or 2.

28. The method of claim 25, wherein at least one of:
the selenium-carrier conjugate kills or permanently inactivates actively growing, inactive, latent or spore-like forms of bacteria;
the selenium-carrier conjugate kills or permanently inactivates fungal infections, wherein the fungi and opportunistic pathogens of the genera Histoplasma, Pneumocystis, Coccidiomyces, Candida; or
the selenium-carrier conjugate kills or permanently inactivates protozoal infections with selenium-carrier conjugates would work by killing protozoal pathogens selected from Plasmodium falciparum, Trypanosoma cruzi, or Entamoeba histolytica.

29. (canceled)

30. (canceled)

31. A method of treating a human or animal patient with a cancer or other disorder caused by abnormal cells comprising:
covalently attaching a synthetic targeting carrier to a selenium compound to form a selenium-carrier conjugate, wherein the synthetic targeting carrier specifically binds to a target on the cancer cell or abnormal cell; and
administering the selenium-carrier conjugate by intravenous, nasal, oral or pulmonary administration to the human or animal patient, wherein the selenium-carrier conjugate reacts in vivo with naturally occurring thiols and oxygen to catalytically generate many short-lived superoxide radicals to treat the cancer or other disorder caused by abnormal cells.

32. The method of claim 31, wherein the abnormal cells are abnormal immune cells that cause an autoimmune disorder selected from multiple sclerosis, lupus or Graves' disease.

33. The method of claim 31, wherein the synthetic targeting carrier is a peptido-mimetic molecule that specifically binds to the targeted cellular plasma membrane protein specific to a cancer cell or another abnormal, disease-causing cell of the patient; or
a peptide precursor of a peptidomimetic synthetic targeting carrier is made by: expressing and displaying a library of peptides having different amino acid sequence permutations on the surface of a bacteriophage, and selecting the bacteriophages that bind to the targeted transmembrane protein on the surface of the one or more membrane-enveloped viruses, wherein the synthesized peptide is attached to Se.

34. (canceled)

35. The method of claim 33, further comprising at least one of:
the step of modifying a selected peptide precursor by at least one of: changing the amino acid sequence, chemical modifying the amino acids, substituting one or more peptide linkages, or substituting with one or more D-amino acids, wherein the modifications may enhance binding affinity of the modified peptide precursor to the target and its resistance to proteolysis, to increase effectiveness of the selenium-carrier conjugate in vivo, and optionally the peptide has SEQ ID NO: 1 or 2; or
the step of selectively ablating specific defective cell types to create niches for replacement cells prior to a stem cell-based cell replacement therapy.

36. (canceled)

37. An antiviral, antibacterial, antifungal, antiprotozoal, anticancer or anti-abnormal human or animal cell method, the method comprising:
targeting a first and a second target of the virus, bacterial, protozoa, cancer or abnormal cell with a first and a second selenium-carrier complex, wherein:
the first target is a viral, bacterial, protozoan, cancer, or abnormal cell targeted by the first selenium-carrier complex; and
the second target is targeted by the second selenium-carrier complex, wherein the Fenton complex is selected from: an organometallic compound or conjugate containing one or more atoms of iron, copper or other transition metals that catalyzes a conversion of superoxide, perhydroxyl radical or hydrogen peroxide into other compounds, that generate hydroxyl radicals and reactive oxygen species; wherein at least a portion of the Fenton Complex is located close to the selenium-carrier complex that catalyzes conversion of selenium-generated superoxide, perhydroxyl radical or hydrogen peroxide into hydroxyl radicals.

38. The method of claim 37, wherein at least one of:
the highly reactive hydroxyl radicals are generated locally from superoxide, perhydroxyl radical, or hydrogen peroxide by metal atoms of the Fenton Complex to chemically modify at least one of: membrane lipids, polypeptides, RNA, DNA, carbohydrates or other biological molecules;
the first and a second selenium-carrier are administered in vivo simultaneously or sequentially;
the Fenton complex is iron, copper or other transition metal covalently attached to, tightly chelated by, coordinated with or enclosed by an organic molecule;
the organic molecule is a synthetic metal-coordinating compound, or a siderophores coupled to the peptide or a peptidomimetic targeting molecule, and optionally the peptide has SEQ ID NO: 1 or 2;
the organo-metallic compound is chemically modified or extended to produce an organic molecule that binds to RNA, DNA or both by intercalate between the bases;
the intercalating organo-metallic compound is membrane-permeable to enveloped viruses to targeting production of hydroxyl radicals and RNA or DNA damaging inactivating and mutagenic effects on the membrane-enveloped virus particles;
the intercalating organo-metallic compound is membrane-impermeable, such that the Fenton complex will preferentially bind to a genetic material of a protein-encapsidated non-enveloped virus particle to target production of hydroxyl radicals to a genetic material of virus;
the viral protein or other molecule that selectively binds to the selenium-carrier complex exists as a dimer, trimer, or higher level multimer, wherein superoxide-generating selenium atoms are positioned within nanometers of Fenton complex metal atoms that convert to highly reactive hydroxyl radicals;
the second selenium-carrier complex of the Fenton complex is directed to a viral binding site different from the peptide or peptidomimetic synthetic carrier that is conjugated to the first selenium-carrier complex;
the first or the second selenium-carrier complex selectively binds to a different, non-competing site on the same target viral protein molecule;
the Fenton complex and the first selenium-carrier complex selectively bind to two different proteins localized to a plasma membrane domain of a virus-infected cell, viral envelope or viral capsid;
the first and second selenium-carrier complexes are formulated for in vivo administration to permit a higher dosage of a relatively lower toxicity iron-based Fenton complex in conjunction with a smaller dosage of the potentially more toxic selenium conjugate, when compared to unconjugated selenium;
the first target is a first viral surface protein, and the second target is a viral membrane envelope or capsid protein;
the Fenton complex is an iron-filled ferritin complex containing roughly 4500 iron atoms brought into the proximity of the active selenium-conjugate by a targeting peptide, peptidomimetic, protein or antibody;
the Fenton complex is constructed by using a chemical linker to covalently cross-link a single ferritin complex to a targeting peptide, peptidomimetic, protein or antibody;
a virus and cell-targeting carrier for the Fenton complex is a peptide, protein, antibody or peptidomimetic further comprising an antibody or peptide that binds selectively to human ferritin protein;
the ferritin is from autologous human plasma;
the first and second targets are pathogens selected bacteria, fungi and protozoans;
the Fenton complex is an organometallic compound or conjugate containing one or more atoms of iron, copper or other transition metals that catalyze conversion of superoxide, perhydroxyl radical or hydrogen peroxide into hydroxyl radicals and other reactive oxygen species;
the Fenton complex metal atoms are highly reactive and chemically modify molecules, including membrane lipids, polypeptides, RNA, DNA, carbohydrates and other biological molecules;
the cancer cells and other abnormal, dysfunctional cells, wherein the first and second selenium-carrier complexes have an increased effect, range, specificity of action, and are synergistic when used in combination comparted to each used individually;
the cancer cells and other abnormal, dysfunctional cells comprise a single surface protein or combinations of cell surface proteins that are distinctive to the cancer cell;
the cancer cells and other abnormal, dysfunctional cells are killed when they are pre-cancerous, senescent, inappropriately secrete signaling molecules, or are otherwise dysfunctional; or
the abnormal cells are immune cells that cause an autoimmune disease, an autoinflammatory disease or an allergy.

39. (canceled)

40. (canceled)

41. (canceled)

42. (canceled)

43. (canceled)

44. (canceled)

45. The method of claim 37, further comprising chemically linking to a peptide or peptidomimetic compound to bind with high selectivity to a protein that is a binding target of the peptide to target selenium to the surface of the virus.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The method of claim 37, wherein the organometallic compound is: [chemical structure]
